(19) United States
(12) Patent Application Publication
(10) Pub. No.: US 2006/0281722 A1
(43) Pub. Date: Dec. 14, 2006

(54) Compounds and uses thereof

(76) Inventors: Michael A. Foley, Chestnut Hill, MA (US); Curtis T. Keith, Boston, MA (US); Ronald T. Borchardt, Lawrence, KS (US)

Correspondence Address: CLARK & ELBING LLP, 101 FEDERAL STREET, BOSTON, MA, 02110, US

(21) Appl. No.: 11/352,846

(22) Filed: Feb. 13, 2006

Related U.S. Application Data
(60) Provisional application No. 60/652,624, filed on Feb. 14, 2005.

(51) Int. Cl. A61K 31/573; A61K 31/553; A61K 31/198

(52) U.S. Cl. 514/171; 514/211.13; 514/567

(57) ABSTRACT

The invention features charge-modified antidepressants and compounds conjugated to either a charged group or a bulky group in a manner that resists in vivo cleavage. The invention provides a method for treating a patient having an inflammatory disease by administering to the patient a compound of the invention.

### CROSS-REFERENCE TO RELATED APPLICATIONS

This application claims benefit from U.S. Provisional Application No. 60/652,624, filed Feb. 14, 2005, hereby incorporated by reference.

### BACKGROUND OF THE INVENTION

The invention relates to the treatment of immunoinflammatory disorders, such as osteoarthritis, Crohn's disease, psoriasis, and rheumatoid arthritis.

The brain is well protected from outside influences by the blood-brain barrier, which prevents the free entry of many circulating molecules, cells, or micro-organisms into the brain interstitial space. However, this is not true for antidepressants, which must penetrate the blood-brain barrier in order to alleviate depression. Thus, in the treatment of peripheral disorders (e.g., psoriasis or arthritis), the brain is exposed to the antidepressant without any therapeutic benefit and with the possibility of severe adverse effects. These adverse effects, which are described in the PDR, include: sedation, nausea, blurry vision, weight gain, erectile dysfunction, night sweats, dizziness, arrhythmias, and angina.

Improved therapies are needed for the treatment of immunoinflammatory disorders.

### SUMMARY OF THE INVENTION

In one aspect, the invention features a conjugate including a compound covalently attached via a linker to a bulky group of greater than 300 daltons or a charged group of less than 300 daltons. The conjugate is described by the formula:
(A)-(L)-(B),
wherein (B) is either a bulky group of greater than 300 daltons or a charged group of less than 300 daltons; (L) is a linker which forms linkage groups with compound (A) and group (B); and (A) is a compound of any of formulas I-VI. Desirably, the conjugate has anti-inflammatory activity in vivo and reduced activity in the central nervous system in comparison to the parent compound.

Conjugates include compounds having formula (A)-(L)-(B) wherein (A) is a compound of formula (I):

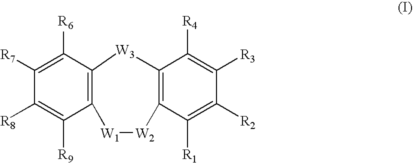

In formula (I), W₃ is O, CHCH₂R₅, or C═CHR₅; W₁-W₂ is OCHR₁₁, SCHR₁₁, N═CR₁₁, CHR₁₀-CHR₁₁, or CR₁₀═CR₁₁; each of R₁, R₂, R₃, R₄, R₆, R₇, R₈, and R₉, is, independently, selected from H, OH, halide, and OG¹; R₅ is CH₂CH₂X₁ or CH(CH₃)CH₂X₁; R₁₀ is H, OH, or OG¹; R₁₁ is H, OH, OG¹, or the group: 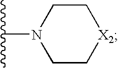
X₁ is NH₂, NHCH₃, N(CH₃)₂, NG¹(CH₃)₂, NG¹CH₃, or NHG¹; X₂ is NH, NCH₃, NG¹CH₃, or NG¹; and G¹ is a bond in a linkage group between (A) and (L), wherein one G¹is present in the comound. In certain embodiments, when (B) is a charged group of less than 300 daltons (B) does not include a carboxylic acid moiety.

In certain embodiments, the conjugate of formula (I) may be further described by formula (II):

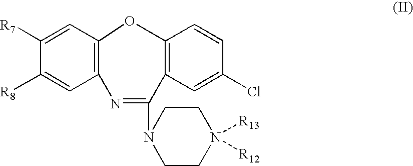

In formula (II), each of R₇ and R₈ is, independently, selected from H, OH, and OG¹; R₁₂ is H, CH₃, or G¹; R₁₃ is CH₃ or absent; and G¹ is a bond in a linkage group between (A) and (L).

In other embodiments, the conjugate of formula (I) may be further described by formula (III):

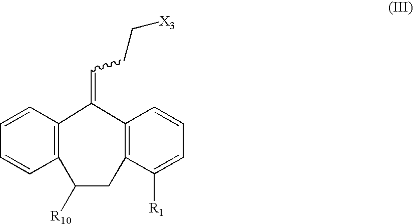

In formula (III), X₃ is NH₂, NHCH₃, N(CH₃)₂, NG¹(CH₃)₂, NG¹CH₃, or NHG¹; each of R₁ and R₁₀ is, independently, selected from H, OH, and OG¹; and G¹ is a bond in a linkage group between (A) and (L).

Conjugates of formula (I) include, for example, morpholine derivatives, such as compound 1:

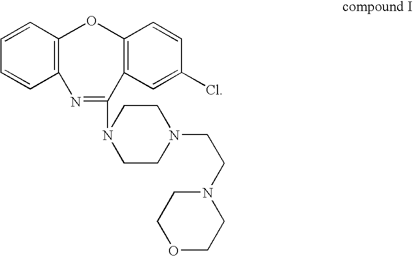

Conjugates of the invention also include compounds having formula (A)-(L)-(B) wherein (A) is a compound of formula (IV):

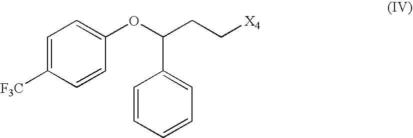

In formula (IV), X₄ is NG¹(CH₃)₂, NG¹CH₃, or NHG¹; and G¹ is a bond in a linkage group between (A) and (L).

Conjugates of the invention also include compounds having formula (A)-(L)-(B) wherein (A) is a comopund of formula (V): 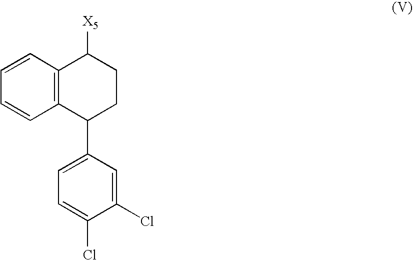
In formula (V), X₅ is NG¹(CH₃)₂, NG¹CH₃, or NHG¹; and G¹ is a bond in a linkage group between (A) and (L).

Conjugates include compounds having formula (A)-(L)-(B) wherein (A) is a compound of formula (VI):

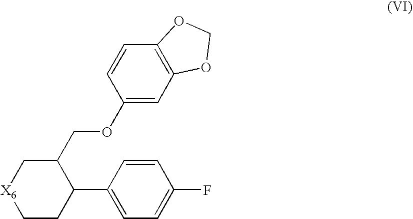

In formula (VI), X₆ is NG¹CH₃, or NG¹; and G¹ is a bond in a linkage group between (A) and (L).

In any of the foregoing compounds, desirably, the linker is described by formula (VII):
G¹-(Z¹)ₒ-(Y¹)ᵤ-(Z²)ₛ-(R₃₀)-(Z³)ₜ-(Y²)ᵥ-(Z⁴)ₚ-G² (VII)

In formula (VII), G¹ is the bond in a linkage group between the compound (A) and the linker; G² is a bond in a linkage group between the linker and the bulky group or between the linker and the charged group; Z¹, Z², Z³, and Z⁴ each, independently, is selected from O, S, and NR₃₁; R₃₁ is hydrogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; Y¹ and Y² are each, independently, selected from carbonyl, thiocarbonyl, sulphonyl, or phosphoryl; o, p, s, t, u, and v are each, independently, 0 or 1; and R₃₀ is a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl, or a chemical bond linking G¹-(Z¹)ₒ-(Y¹)ᵤ-(Z²)ₛ- to -(Z³)ₜ-(Y²)ᵥ-(Z⁴)ₚ-G².

The bulky group can be a naturally occurring polymer or a synthetic polymer. Examples of natural polymers that can be used include, without limitation, glycoproteins, polypeptides, or polysaccharides. Examples of synthetic polymers that can be used as bulky groups include, without limitation, polyethylene glycol and the synthetic polypetide N-hxg. The bulky group may also include a corticosteroid. Desirably, the corticosteroid is selected from hydrocortisone, methylprednisolone, prednisolone, prednisone, dexamethasone, budesonide, and triamcinolone.

The charged group can be a cation or an anion. Desirably, the charged group is a polyanion including at least two negatively charged moieties or a cation having at least one positively charged moiety. Desirably the charged group includes two, three, or four charged moieties. Exemplary charged groups include a morpholine ring system, which is cationic at physiological pH.

The invention also features a charge-modified antidepressant including a parent antidepressant having an amino nitrogen that has been converted to a quaternary amino group or guanidinium group. Desirably, the charge-modified antidepressant has anti-inflammatory activity in vivo and, more desirably, reduced activity in the central nervous system in comparison to the parent antidepressant.

In any of the compositions, methods and kits described herein, desirably the parent antidepressant is a tricyclic antidepressant, a selective serotonin reuptake inhibitor, or a serotonin norepinephrine reuptake inhibitor.

Charge-modified antidepressants include compounds of formula (VIII):

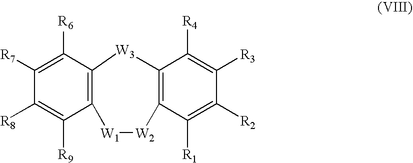

In formula (VIII), W₃ is O, CHCH₂R₅, or C═CHR₅; W₁-W₂ is OCHR₁₁, SCHR₁₁, N═CR₁₁, CHR₁₀—CHR₁₁, or CR₁₀═CR₁₁; each of R₁, R₂, R₃, R₄, R₆, R₇, R₈, and R₉, is, independently, selected from H, OH, and halide; R₅ is CH₂CH₂X₁ or CH(CH₃)CH₂X₁; R₁₀ is H or OH; R₁₁ is H, OH, or the group: 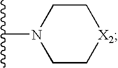
X₁ is NH₂, NHCH₃, N(CH₃)₂, NR₁₄R₁₅R₁₆, or NR₁₇X₇; X₂ is NH, NCH₃, NR₂₁R₂₂, or NX₇; each of R₁₄, R₁₅, R₁₆, R₂₁, and R₂₂ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl; R₁₇ is H or CH₃; X₇ is 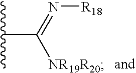
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

In certain embodiments, the charge-modified antidepressant of formula (VIII) can further be described by formula (IX):

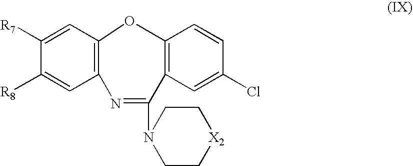

In formula (IX), each of R₇ and R₈ is, independently, selected from H, and OH; X₂ is NR₂₁R₂₂, or NX₇; each of R₂₁, and R₂₂ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl; X₇ is 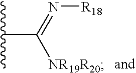
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

In other embodiments, the charge-modified antidepressant of formula (VIII) can further be described by formula (X):

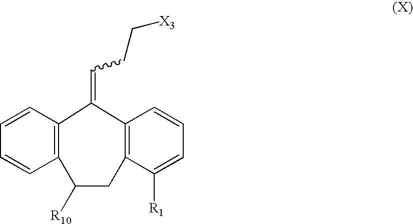

In formula (X), each of R₁ and R₁₀ is, independently, selected from H, and OH; X₃ is NR₁₄R₁₅R₁₆, or NR₁₇X₇; each of R₁₄, R₁₅, and R₁₆ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl; R₁₇ is H or CH₃; X₇ is 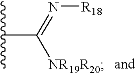
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

Charge-modified antidepressants of the invention also include compounds of formula (XI):

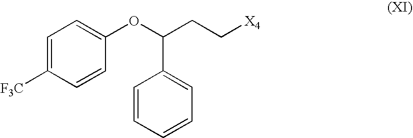

In formula (XI), X₄ is NR₁₄R₁₅R₁₆, or NR₁₇X₇; each of R₁₄, R₁₅, and R₁₆ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl; R₁₇ is H or CH₃; X₇ is 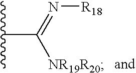
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

Charge-modified antidepressants of the invention also include compounds of formula (XII):

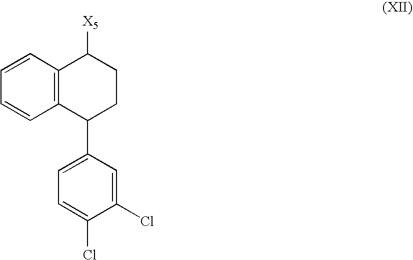

In formula (XII), X₅ is NR₁₄R₁₅R₁₆, or NR₁₇X₇; each of R₁₄, R₁₅, and R₁₆ is, independently, selected from C₁₋₄alkyl, C₂₋₆ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl; R₁₇ is H or CH₃; X₇ is 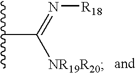
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

Yet other charge-modified antidepressants of the invention are compounds of formula (XIII):

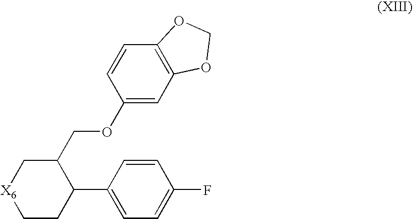

In formula (XIII), X₆ is NR₂₁R₂₂, or NX₇; each of R₂₁, and R₂₂ is, independently, selected from C₁₋₄alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl; X₇ is 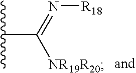
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

The invention also features a method for suppressing secretion of one or more proinflammatory cytokines in a patient in need thereof by administering to the patient a conjugate or charge-modified antidepressant of the invention in an amount sufficient to suppress secretion of proinflammatory cytokines in the patient.

The invention also features a method for treating a patient diagnosed with an immunoinflammatory disorder by administering to the patient a conjugate or charge-modified antidepressant of the invention in an amount sufficient to treat said patient.

Immunoinflammatory disorders that can be treated by administering to a patient a conjugate or charge-modified antidepressant described herein include, without limitation, rheumatoid arthritis, Crohn's disease, ulcerative colitis, asthma, osteoarthritis, chronic obstructive pulmonary disease, polymyalgia rheumatica, giant cell arteritis, systemic lupus erythematosus, atopic dermatitis, multiple sclerosis, myasthenia gravis, psoriasis, ankylosing spondylitis, or psoriatic arthritis.

The invention also features a method for treating a patient diagnosed with an immunoinflammatory disorder selected from rheumatoid arthritis, osteoarthritis, Crohn's disease, ulcerative colitis, asthma, chronic obstructive pulmonary disease, polymyalgia rheumatica, giant cell arteritis, systemic lupus erythematosus, atopic dermatitis, multiple sclerosis, myasthenia gravis, psoriasis, ankylosing spondylitis, and psoriatic arthritis. This method includes the step of administering to the patient a compound having the formula: 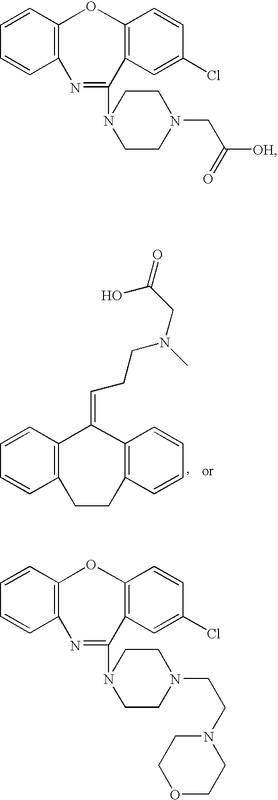
in an amount sufficient to treat said patient.

The invention features a method for inhibiting passage across the blood-brain barrier of a compound by covalent attachment of a bulky group of greater than 300 daltons or a charged group of less than 300 daltons. Desirably, the group increases the size, or alters the charge, of the compound sufficiently to inhibit passage across the blood-brain barrier without destroying the anti-inflammatory activity of the compound covalently attached to the group. Desirably, the covalent attachment is resistant to in vivo cleavage, further protecting the brain from CNS active metabolites. The bulky group or charged group can be attached via a nitrogen atom present in the parent compound.

The invention features a method for inhibiting passage across the blood-brain barrier of an antidepressant having an amine nitrogen by converting the amine nitrogen to a quaternary amino group or guanidinium group. The group alters the charge of the antidepressant sufficiently to inhibit passage across the blood-brain barrier without destroying the anti-inflammatory activity of said antidepressant.

The invention features a pharmaceutical composition that includes an effective amount of a conjugate or charge-modified antidepressant described herein in any pharmaceutically acceptable form, along with a pharmaceutically acceptable carrier or diluent.

The invention features a pharmaceutical composition that includes a conjugate or charge-modified antidepressant and a corticosteroid in amounts that together are sufficient to treat an immunoinflammatory disorder in a patient in need thereof.

The invention further features a pharmaceutical composition that includes a a compound having formula: 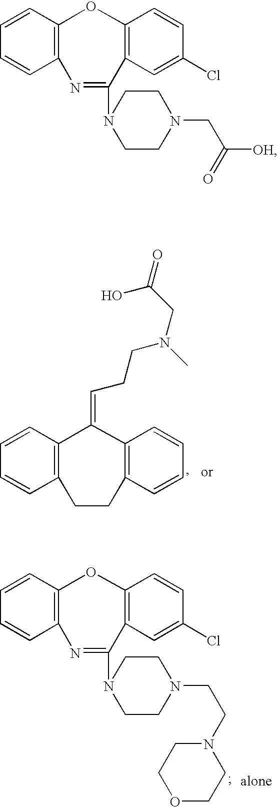
or in combination with a corticosteroid, wherein the compound and the corticosteroid are present in an amount, that together, is sufficient to treat an immunoinflammatory disorder when administered to a patient.

If desired, the above pharmaceutical compositions may include one or more additional compounds (e.g., a glucocorticoid receptor modulator, NSAID, COX-2 inhibitor, DMARD, biologic, small molecule immunomodulator, xanthine, anticholinergic compound, beta receptor agonist, bronchodilator, non-steroidal immunophilin-dependent immunosuppressant, vitamin D analog, psoralen, retinoid, or 5-amino salicylic acid). The composition may be formulated, for example, for topical administration or systemic administration.

The invention also features a method for treating a patient diagnosed with or at risk of developing an immunoinflammatory disorder by administering to the patient a conjugate or charge-modified antidepressant and a corticosteroid simultaneously or within 14 days of each other in amounts that together are sufficient to treat the patient.

The invention further features a method of modulating an immune response (e.g., by decreasing proinflammatory cytokine secretion or production, or by modulating adhesion, gene expression, chemokine secretion, presentation of MHC complex, presentation of costimulation signals, or cell surface expression of other mediators) in a patient by administering to the patient a conjugate or charge-modified antidepressant and a corticosteroid simultaneously or within 14 days of each other in amounts that together are sufficient to modulate the immune response in the patient.

The invention also features a method for treating a patient diagnosed with or at risk of developing an immunoinflammatory disorder selected from rheumatoid arthritis, osteoarthritis, Crohn's disease, ulcerative colitis, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, and psoriatic arthritis. The method includes administering to the patient:

(i) a compound having the formula: 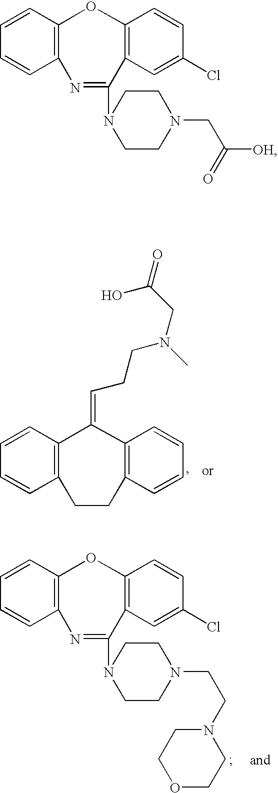
(ii) a corticosteroid,
wherein the compound and the corticosteroid are administered simultaneously or within 14 days of each other in an amount, that together, is sufficient to treat the patient.

In either of the foregoing methods, the patient may also be administered one or more additional compounds (e.g., a glucocorticoid receptor modulator, NSAID, COX-2 inhibitor, DMARD, biologic, small molecule immunomodulator, xanthine, anticholinergic compound, beta receptor agonist, bronchodilator, non-steroidal immunophilin-dependent immunosuppressant, vitamin D analog, psoralen, retinoid, or 5-amino salicylic acid).

If desired, the conjugate or charge-modified antidepressant and/or corticosteroid may be administered in a low dosage or a high dosage. The drugs are desirably administered within 10 days of each other, more desirably within five days of each other, and even more desirably within twenty-four hours of each other or even simultaneously (i.e., concomitantly).

The invention features a method for treating an immunoinflammatory disorder in a patient in need thereof by concomitantly administering to the patient a conjugate or charge-modified antidepressant and a corticosteroid in amounts that together are more effective in treating the immunoinflammatory disorder than the administration of the corticosteroid in the absence of the conjugate or charge-modified antidepressant.

The invention also features a method for treating an immunoinflammatory disorder in a patient in need thereof by concomitantly administering to the patient a conjugate or charge-modified antidepressant and a corticosteroid in amounts that together are more effective in treating the immunoinflammatory disorder than the administration of the conjugate or charge-modified antidepressant in the absence of the corticosteroid.

The invention further features a method for treating an immunoinflammatory disorder in a patient in need thereof by administering a corticosteroid to the patient; and administering a conjugate or charge-modified antidepressant to the patient; wherein: (i) the corticosteroid and conjugate or charge-modified antidepressant are concomitantly administered and (ii) the respective amounts of the corticosteroid and the conjugate or charge-modified antidepressant administered to the patient are more effective in treating the immunoinflammatory disorder compared to the administration of the corticosteroid in the absence of the conjugate or charge-modified antidepressant or the administration of the conjugate or charge-modified antidepressant in the absence of the corticosteroid.

The invention also features a pharmaceutical composition in unit dose form, the composition including a corticosteroid; and a conjugate or charge-modified antidepressant, wherein the amounts of the corticosteroid and the conjugate or charge-modified antidepressant, when administered to the patient, are more effective in treating the immunoinflammatory disorder compared to the administration of the corticosteroid in the absence of the conjugate or charge-modified antidepressant or the administration of the conjugate or charge-modified antidepressant in the absence of the corticosteroid.

The invention features a kit that includes (i) a composition that includes a conjugate or charge-modified antidepressant and a corticosteroid; and (ii) instructions for administering the composition to a patient diagnosed with an immunoinflammatory disorder.

The invention features a kit that includes: (i) a conjugate or charge-modified antidepressant; (ii) a corticosteroid; and (iii) instructions for administering the conjugate or charge-modified antidepressant and the corticosteroid to a patient diagnosed with an immunoinflammatory disorder.

The invention also features a kit that includes (i) a conjugate or charge-modified antidepressant; and (ii) instructions for administering the conjugate or charge-modified antidepressant and a corticosteroid to a patient diagnosed with or at risk of developing an immunoinflammatory disorder.

The invention features a kit that includes: (i) a compound having the formula: 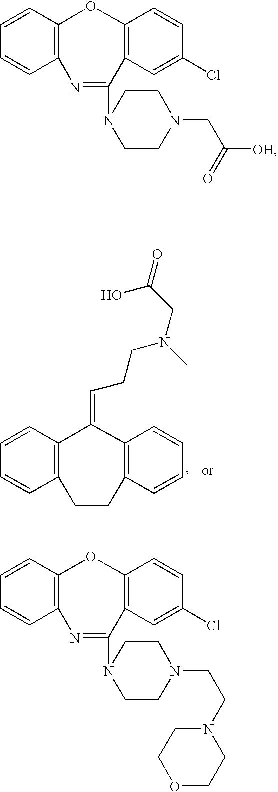
(ii) a corticosteroid; and (iii) instructions for systemically administering the compound and the corticosteroid to a patient diagnosed with or at risk of developing an immunoinflammatory disorder.

The invention further features a kit that includes: (i) a compound having the formula: 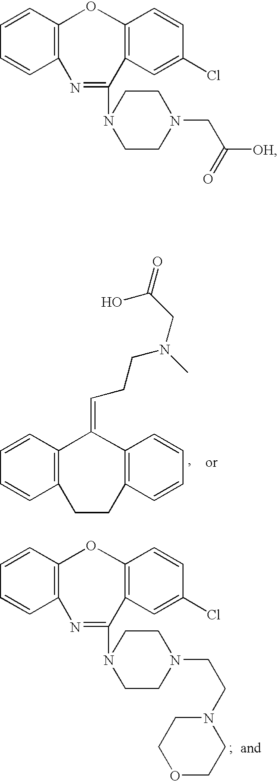
(ii) instructions for administering said compound to a patient diagnosed with an immunoinflammatory disorder selected from rheumatoid arthritis, osteoarthritis, Crohn's disease, ulcerative colitis, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, and psoriatic arthritis.

If desired, the corticosteroid can be replaced in the methods, compositions, and kits of the invention with a glucocorticoid receptor modulator or other steroid receptor modulator.

Thus, in another aspect, the invention features a composition that includes a conjugate or charge-modified antidepressant and a glucocorticoid receptor modulator in amounts that together are sufficient to treat an immunoinflammatory disorder in a patient in need thereof. If desired, the composition may include one or more additional compounds. The composition may be formulated, for example, for topical administration or systemic administration.

The invention features a method for treating a patient diagnosed with or at risk of developing an immunoinflammatory disorder by administering to the patient a conjugate or charge-modified antidepressant and a glucocorticoid receptor modulator simultaneously or within 14 days of each other in amounts that together are sufficient to treat the patient. The drugs are desirably administered within 10 days of each other, more desirably within five days of each other, and even more desirably within twenty-four hours of each other or even simultaneously (i.e., concomitantly).

The invention also features a method of modulating an immune response (e.g., by decreasing proinflammatory cytokine secretion or production, or by modulating adhesion, gene expression, chemokine secretion, presentation of MHC complex, presentation of costimulation signals, or cell surface expression of other mediators) in a patient by administering to the patient a conjugate or charge-modified antidepressant and a glucocorticoid receptor modulator simultaneously or within 14 days of each other in amounts that together are sufficient to modulate the immune response in the patient.

In a related aspect, the invention features a method for treating an immunoinflammatory disorder in a patient in need thereof by concomitantly administering to the patient a conjugate or charge-modified antidepressant and a glucocorticoid receptor modulator in amounts that together are more effective in treating the immunoinflammatory disorder than the administration of the glucocorticoid receptor modulator in the absence of the conjugate or charge-modified antidepressant.

In yet another related aspect, the invention features a method for treating an immunoinflammatory disorder in a patient in need thereof by concomitantly administering to the patient a conjugate or charge-modified antidepressant and a glucocorticoid receptor modulator in amounts that together are more effective in treating the immunoinflammatory disorder than the administration of the conjugate or charge-modified antidepressant in the absence of the glucocorticoid receptor modulator.

In still another related aspect, the invention features a method for treating an immunoinflammatory disorder in a patient in need thereof by administering a glucocorticoid receptor modulator to the patient; and administering a conjugate or charge-modified antidepressant to the patient; wherein: (i) the glucocorticoid receptor modulator and conjugate or charge-modified antidepressant are concomitantly administered and (ii) the respective amounts of the glucocorticoid receptor modulator and the conjugate or charge-modified antidepressant administered to the patient are more effective in treating the immunoinflammatory disorder compared to the administration of the glucocorticoid receptor modulator in the absence of the conjugate or charge-modified antidepressant or the administration of the conjugate or charge-modified antidepressant in the absence of the glucocorticoid receptor modulator.

The invention also features a pharmaceutical composition in unit dose form, the composition including a glucocorticoid receptor modulator; and a conjugate or charge-modified antidepressant of the invention, wherein the amounts of the glucocorticoid receptor modulator and the conjugate or charge-modified antidepressant, when administered to the patient, are more effective in treating the immunoinflammatory disorder compared to the administration of the glucocorticoid receptor modulator in the absence of the conjugate or charge-modified antidepressant or the administration of the conjugate or charge-modified antidepressant in the absence of the glucocorticoid receptor modulator.

The invention also features a kit that includes (i) a composition that includes a conjugate or charge-modified antidepressant of the invention and a glucocorticoid receptor modulator; and (ii) instructions for administering the composition to a patient diagnosed with an immunoinflammatory disorder.

In a related aspect, the invention features a kit that includes: (i) a conjugate or charge-modified antidepressant of the invention; (ii) a glucocorticoid receptor modulator; and (iii) instructions for administering the conjugate or charge-modified antidepressant and the glucocorticoid receptor modulator to a patient diagnosed with an immunoinflammatory disorder.

In a related aspect, the invention features a kit that includes (i) a conjugate or charge-modified antidepressant of the invention; and (ii) instructions for administering the conjugate or charge-modified antidepressant and a second compound selected from the group consisting of a glucocorticoid receptor modulator, small molecule immunomodulator, xanthine, anticholinergic compound, biologic, NSAID, DMARD, COX-2 inhibitor, beta receptor agonist, bronchodilator, non-steroidal immunophilin-dependent immunosuppressant, vitamin D analog, psoralen, retinoid, and 5-amino salicylic acid to a patient diagnosed with or at risk of developing an immunoinflammatory disorder.

In another aspect, the invention features a pharmaceutical composition that includes a conjugate or charge-modified antidepressant of the invention and a second compound selected from the group consisting of a glucocorticoid receptor modulator, NSAID, COX-2 inhibitor, DMARD, biologic, small molecule immunomodulator, xanthine, anticholinergic compound, beta receptor agonist, bronchodilator, non-steroidal immunophilin-dependent immunosuppressant, vitamin D analog, psoralen, retinoid, and 5-amino salicylic acid.

The invention features another kit that includes (i) a corticosteroid; and (ii) instructions for administering said corticosteroid and a conjugate or charge-modified antidepressant of the invention to a patient diagnosed with or at risk of developing an immunoinflammatory disorder.

The invention also features methods for identifying compounds or combinations of compounds that may be useful for modulating an immune response (e.g., by decreasing proinflammatory cytokine secretion or production, or by modulating adhesion, gene expression, chemokine secretion, presentation of MHC complex, presentation of costimulation signals, or cell surface expression of other mediators). One such method includes the steps of: (a) contacting cells in vitro with a conjugate or charge-modified antidepressant and a candidate compound; and (b) determining whether the combination of the conjugate or charge-modified antidepressant and the candidate compound reduces proinflammatory cytokine secretion relative to cells contacted with the conjugate or charge-modified antidepressant but not contacted with the candidate compound or cells contacted with the candidate compound but not with the conjugate or charge-modified antidepressant. A modulation of proinflammatory cytokine secretion or production, adhesion, gene expression, chemokine secretion, presentation of MHC complex, presentation of costimulation signals, or cell surface expression of other mediators) identifies the combination as a combination that is useful for treating a patient in need of such treatment.

In another aspect, the invention features a method for identifying a combination that may be useful for the treatment of an immunoinflammatory disorder by: (a) identifying a compound that modulates the immune response; (b) contacting proliferating cells in vitro with a conjugate or charge-modified antidepressant and the compound identified in step (a); and (c) determining whether the combination of the conjugate or charge-modified antidepressant and the compound identified in step (a) modulates the immune response, relative to immune response of cells contacted with the conjugate or charge-modified antidepressant but not contacted with the compound identified in step (a) or contacted with the compound identified in step (a) but not contacted with the conjugate or charge-modified antidepressant. A modulation in the immune response (e.g., a reduction in the production or secretion of proinflammatory cytokines) identifies the combination as a combination that may be useful for the treatment of an immunoinflammatory disorder.

The invention also features a method for identifying combinations of compounds useful for suppressing the secretion of proinflammatory cytokines in a patient in need of such treatment by: (a) contacting cells in vitro with a conjugate or charge-modified antidepressant and a candidate compound; and (b) determining whether the combination of the conjugate or charge-modified antidepressant and the candidate compound reduces cytokine levels in blood cells stimulated to secrete the cytokines relative to cells contacted with the conjugate or charge-modified antidepressant but not contacted with the candidate compound or cells contacted with the candidate compound but not with the conjugate or charge-modified antidepressant, wherein a reduction of the cytokine levels identifies the combination as a combination that is useful for treating a patient in need of such treatment.

Compounds useful in the invention include those described herein in any of their pharmaceutically acceptable forms, including isomers such as diastereomers and enantiomers, salts, esters, solvates, and polymorphs thereof, as well as racemic mixtures and pure isomers of the compounds described herein.

The methods and compositions described herein can also be used to generate information useful, for example, for increasing investment in a company or increasing consumer demand for the methods and/or compositions.

The invention therefore features a method of increasing consumer demand for a pharmaceutical composition (e.g., the articles of the invention) or therapeutic regimen (e.g., the administration of articles of the invention) described herein. The method includes the step of disseminating information about the pharmaceutical composition or therapeutic regimen.

The invention further features a method of increasing investment in a company seeking governmental approval for the sale of a pharmaceutical composition and/or therapeutic regimen described herein. The method includes the steps of i) disseminating information about the pharmaceutical composition or therapeutic regimen and ii) disseminating information about the intent of the company to market the pharmaceutical composition or therapeutic regimen.

Consumer demand for a pharmaceutical composition described herein can be increased by disseminating information about the utility, efficacy, or safety of the pharmaceutical composition. Consumers include health maintenance organizations, hospitals, doctors, and patients. Typically, the information will be disseminated prior to a governmental approval for the sale of a composition or therapeutic regimen of the invention.

A company planning to sell a pharmaceutical composition described herein can increase investment therein by disseminating information about the company's intention to seek governmental approval for the sale of and disseminating information about the pharmaceutical composition and/or therapeutic regimen of the invention. For example, the company can increase investment by disseminating information about in vivo studies conducted, or planned, by the company, including, without limitation, information about the toxicity, efficacy, or dosing requirements of a pharmaceutical composition or therapeutic regimen of the invention. The company can also increase investment by disseminating information about the projected date of governmental approval of a pharmaceutical composition or therapeutic regimen of the invention.

Information can be disseminated in any of a variety of ways, including, without limitation, by press release, public presentation (e.g., an oral or poster presentation at a trade show or convention), on-line posting at a web site, and mailing. Information about the pharmaceutical composition or therapeutic regimen can include, without limitation, a structure, diagram, figure, chemical name, common name, tradename, formula, reference label, or any other identifier that conveys the identity of the pharmaceutical composition or therapeutic regimen of the invention to a person.

By “in vivo studies” is meant any study in which a pharmaceutical composition or therapeutic regimen of the invention is administered to a mammal, including, without limitation, non-clinical studies, e.g., to collect data concerning toxicity and efficacy, and clinical studies.

By “projected date of governmental approval” is meant any estimate of the date on which a company will receive approval from a governmental agency to sell, e.g., to patients, doctors, or hospitals, a pharmaceutical composition or therapeutic regimen of the invention. A governmental approval includes, for example, the approval of a drug application by the Food and Drug Administration, among others.

In the generic descriptions of compounds of this invention, the number of atoms of a particular type in a substituent group is generally given as a range, e.g., an alkyl group containing from 1 to 4 carbon atoms or C₁₋₄ alkyl. Reference to such a range is intended to include specific references to groups having each of the integer number of atoms within the specified range. For example, an alkyl group from 1 to 4 carbon atoms includes each of C₁, C₂, C₃, and C₄. A C₁₋₁₂ heteroalkyl, for example, includes from 1 to 12 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups.

By “C₁₋₄ alkyl” is meant a a branched or unbranched hydrocarbon group having from 1 to 4 carbon atoms. A C₁₋₄ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₄ alkyls include, without limitation, methyl, ethyl, n-propyl, isopropyl, cyclopropyl, cyclopropylmethyl, n-butyl, iso-butyl, sec-butyl, tert-butyl, and cyclobutyl.

By “C₂₋₄ alkenyl” is meant a branched or unbranched hydrocarbon group containing one or more double bonds and having from 2 to 4 carbon atoms. A C₂₋₄ alkenyl may optionally include monocyclic or polycyclic rings, in which each ring desirably has from three to six members. The C₂₋₄ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₄ alkenyls include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl.

By “C₂₋₄ alkynyl” is meant a branched or unbranched hydrocarbon group containing one or more triple bonds and having from 2 to 4 carbon atoms. A C₂₋₄ alkynyl may optionally include monocyclic, bicyclic, or tricyclic rings, in which each ring desirably has five or six members. The C₂₋₄ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₄ alkynyls include, without limitation, ethynyl, 1-propynyl, 2-propynyl, 1-butynyl, 2-butynyl, and 3-butynyl.

By “C₂₋₆ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. Examples of C₁₋₇ heteroalkyls include, without limitation, methoxymethyl and ethoxyethyl.

By “halide” is meant bromine, chlorine, iodine, or fluorine.

By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C-₁₋₇ heteroalkyl.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl, heteroalkyl, heteroaryl, and/or aryl groups, resulting in a positive charge at the nitrogen atom.

As used herein, the term “treating” refers to administering a pharmaceutical composition for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering treatment to a patient already suffering from a disease to improve the patient's condition. Thus, in the claims and embodiments, treating is the administration to a mammal either for therapeutic or prophylactic purposes.

The term “administration” or “administering” refers to a method of giving a dosage of a pharmaceutical composition to a mammal, wherein the conjugate or charge-modified antidepressant is administered by a route selected from, without limitation, inhalation, ocular, parenteral, dermal, transdermal, buccal, rectal, sublingual, perilingual, nasal, topical administration and oral administration. Parenteral administration includes intravenous, intraperitoneal, subcutaneous, and intramuscular administration. The preferred method of administration can vary depending on various factors, e.g., the components of the pharmaceutical composition, site of the potential or actual disease and severity of disease.

By “parent antidepressant” is meant the antidepressant which is modified by conjugation to a bulky group or a charged group, or the antidepressant which is modified by conversion of an amine nitrogen present in the parent antidepressant to a quaternary amino group or a guanidinium group.

As used herein, “charge-modified antidepressant” means a parent antidepressant bearing an amino nitrogen which has been converted to a quaternary amino group or guanidinium group. Desirably, the charge-modified antidepressant has anti-inflammatory activity in vivo and reduced activity in the central nervous system in comparison to the parent antidepressant. Charge-modified antidepressants need not exhibit any antidepressant activity. In many instances, owing to their altered biodistribution, a modified antidepressant will exhibit little or no antidepressant activity in vivo.

By “reduced CNS activity” for a conjugate or charge-modified antidepressant is meant that the ratio of AUC_{brain} (area under the curve in brain tissue) to AUC_{blood} (area under the curves in whole blood) is reduced for the conjugate or charge-modified antidepressant in comparison to the parent antidepressant administered under the same conditions. The AUC calculation includes the administered compound and any metabolites, having anti-inflammatory activity, thereof.

By “charged moiety” is meant a moiety which loses a proton at physiological pH thereby becoming negatively charged (e.g., carboxylate, or phosphodiester), a moiety which gains a proton at physiological pH thereby becoming positively charged (e.g., ammonium, guanidinium, or amidinium), a moiety that includes a net formal positive charge without protonation (e.g., quaternary ammonium), or a moiety that includes a net formal negative charge without loss of a proton (e.g., borate, BR₄⁻).

By “resistant to in vivo cleavage” is meant that, in vivo, less than 30, 20, 10, 5, 2, or 1 percent of the administered drug is cleaved, e.g., separating the antidepressant from the charged group or the bulky group, prior to excretion.

By “an amount sufficient” is meant the amount of a compound of the invention required to treat or prevent an immunoinflammatory disease in a clinically relevant manner. A sufficient amount of an active compound used to practice the present invention for therapeutic treatment of conditions caused by or contributing to an immunoinflammatory disease varies depending upon the manner of administration, the age, body weight, and general health of the patient. Ultimately, the prescribers will decide the appropriate amount and dosage regimen. The appropriate amounts for any monotherapy or combination therapy described herein can be determined from animal models, in vitro assays, and/or clinical studies.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, osteoarthritis; atherosclerosis; atopic dermatitis; autoimmune hemolytic anemia; autoimmune hepatitis; Behcet's disease; Bell's palsy; bullous pemphigoid; cerebral ischaemia; chronic obstructive pulmonary disease; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; inflammatory bowel or gastrointestinal disorders, inflammatory dermatoses; lichen planus; lupus nephritis; lymphomatous tracheobronchitis; macular edema; multiple sclerosis; myasthenia gravis; myositis; osteoarthritis; pancreatitis; pemphigoid gestationis; pemphigus vulgaris; polyarteritis nodosa; polymyalgia rheumatica; pruritus scroti; pruritis/inflammation, psoriasis; psoriatic arthritis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; tuberculosis; type-i diabetes; ulcerative colitis; uveitis; vasculitis; and Wegener's granulomatosis.

By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis. As will be appreciated by one skilled in the art, a particular disease, disorder, or condition may be characterized as being both a proliferative skin disease and an inflammatory dermatosis. An example of such a disease is psoriasis.

By “treating” is meant administering or prescribing a pharmaceutical composition for the treatment or prevention of an immunoinflammatory disease.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds. In one embodiment of the invention, the patient subject to a treatment described herein does not have clinical depression, an anxiety or panic disorder, an obsessive/compulsive disorder, alcoholism, an eating disorder, an attention-deficit disorder, a borderline personality disorder, a sleep disorder, a headache, premenstrual syndrome, an irregular heartbeat, schizophrenia, Tourette's syndrome, or phobias.

By “SSRI” is meant any member of the class of compounds that (i) inhibit the uptake of serotonin by neurons of the central nervous system, (ii) have an inhibition constant (Ki) of 10 nM or less, and (iii) a selectivity for serotonin over norepinephrine (i.e., the ratio of Ki(norepinephrine) over Ki(serotonin)) of greater than 100. Typically, SSRIs are administered in dosages of greater than 10 mg per day when used as antidepressants. Exemplary SSRIs for use in the invention are described herein.

For any reference provided herein to a numbered position in a tricylic antidepressant and related compounds, the recited position is defined by the numbering scheme below, wherein W₁, W₂, and W₃ are as defined in formula XIV.

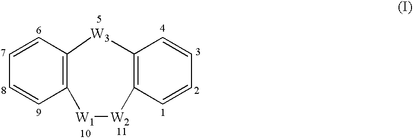

The invention features conjugates and charge-modified antidepressants useful for the treatment of inflammatory diseases, such as osteoarthritis, rheumatoid arthritis, and psoriasis, among others. Desirably, the compounds of the invention have reduced CNS activity in comparison to their parent antidepressants. As a result, compounds of the invention can be used for the treatment of inflammatory conditions, but with reduced CNS side effects.

By “corticosteroid” is meant any naturally occurring or synthetic compound characterized by a hydrogenated cyclopentanoperhydro-phenanthrene ring system and having immunosuppressive and/or antinflammatory activity. Naturally occurring corticosteriods are generally produced by the adrenal cortex. Synthetic corticosteroids may be halogenated. Examples corticosteroids are provided herein.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Examplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of corticosteroid formulated for administration by inhalation will differ from a low dosage of corticosteroid formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By a “moderate dosage” is meant the dosage between the low dosage and the high dosage.

By a “dosage equivalent to a prednisolone dosage” is meant a dosage of a corticosteroid that, in combination with a given dosage of a conjugate or charge-modified antidepressant produces the same anti-inflammatory effect in a patient as a dosage of prednisolone in combination with that dosage.

By “more effective” is meant that a method, composition, or kit exhibits greater efficacy, is less toxic, safer, more convenient, better tolerated, or less expensive, or provides more treatment satisfaction than another method, composition, or kit with which it is being compared. Efficacy may be measured by a skilled practitioner using any standard method that is appropriate for a given indication.

By “sustained release” or “controlled release” is meant that the therapeutically active component is released from the formulation at a controlled rate such that therapeutically beneficial blood levels (but below toxic levels) of the component are maintained over an extended period of time ranging from e.g., about 12 to about 24 hours, thus, providing, for example, a 12 hour or a 24 hour dosage form.

The term “linkage group” refers to the covalent bond that results from the combination of reactive moieties of linker (L) with functional groups of (A) or (B). Examples of linkage groups include, without limitation, ester, carbamate, thioester, imine, disulfide, amine, amide, ether, thioether, sulfonamide, isourea, isothiourea, imidoester, amidine, phosphoramidate, phosphodiester, and thioether.

Other features and advantages of the invention will be apparent from the following Detailed Description and the claims.

### DETAILED DESCRIPTION

The invention features peripherally acting conjugates and charge-modified antidepressants which have reduced CNS activity in comparison their parent antidepressants. The conjugates described herein have three characteristic components: a compound covalently tethered, via a linker, to a group that is bulky or charged. The charge-modified antidepressants described herein are antidepressants structurally modified to include a charge.

Antidepressants

Compounds which can be modified to inhibit passage across the blood-brain barrier include, without limitation, tricyclic antidepressants, selective serotonin reuptake inhibitors, and serotonin norepinephrine reuptake inhibitors. The structures of antidepressants useful in the methods and compositions of the invention are provided below. These are structural examples of parent antidepressants which can be modified as described herein to achieve a reduction in CNS activity. Conjugates of the invention may be prepared by modification of an available functional group present in an antidepressant (e.g., an amino or hydroxyl group). Alternatively, an alkyl group can be removed from a parent antidepressant, e.g., to form a primary or secondary amine, prior to either conjugation with a bulky group or a charged group, or chemical conversion to a quaternary amino group or guanidinium group.

Tricyclic Antidepressants (TCAs)

TCAs are antidepressant compounds having the formula (XIV):

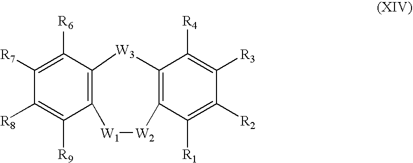

In formula (XIV), W₃ is O, CHCH₂R₅, or C═CHR₅; W₁—W₂ is OCHR₁₁, SCHR₁₁, N═CR₁₁, CHR₁₀—CHR₁₁, or CR₁₀═CR ₁₁; each of R₁, R₂, R₃, R₄, R₆, R₇, R₈, and R₉, is, independently, selected from H, OH, and halide; R₅ is CH₂CH₂X₁ or CH(CH₃)CH₂X₁; R₁₀ is H or OH; R₁₁ is H, OH, or the group: 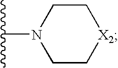
X₁ is NH₂, NHCH₃, N(CH₃)₂; and X₂ is NH or NCH₃.

Exemplary tricyclic antidepressants include, amoxapine, 8-hydroxyamoxapine, 7-hydroxyamoxapine, loxapine, 8-hydroxyloxapine, amitriptyline, 10-hydroxyamitriptyline (E and Z isomers), 3-hydroxyamitriptyline, 2-hydroxyamitriptyline, clomipramine, 8-hydroxychloripramine, 11-hydroxyclomipramine, 8-hydroxydesmethhylclomipramine, didesmethylclomipramine and 2-hydroxydesmethylclomipramine, doxepin, 2-hydroxydoxepin, 7-hydroxydoxepin, 8-hydroxydoxepin, 9-hydroxydoxepin, imipramine, 2-hydroxyimipramine, trimipramine, desipramine, 2-hydroxydesipramine, nortriptyline, 1-hydroxynortriptyline (E and Z isomers), 10-hydroxynortriptyline (E and Z isomers), 2-hydroxyprotripyline, and protriptyline. The structures of some of these compounds are provided below.

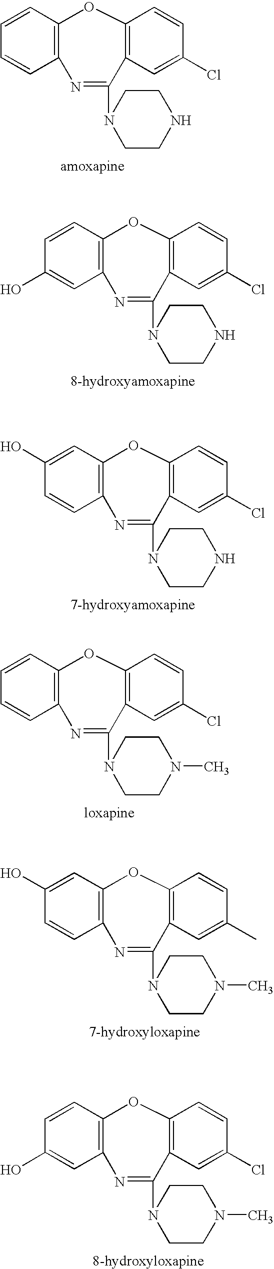

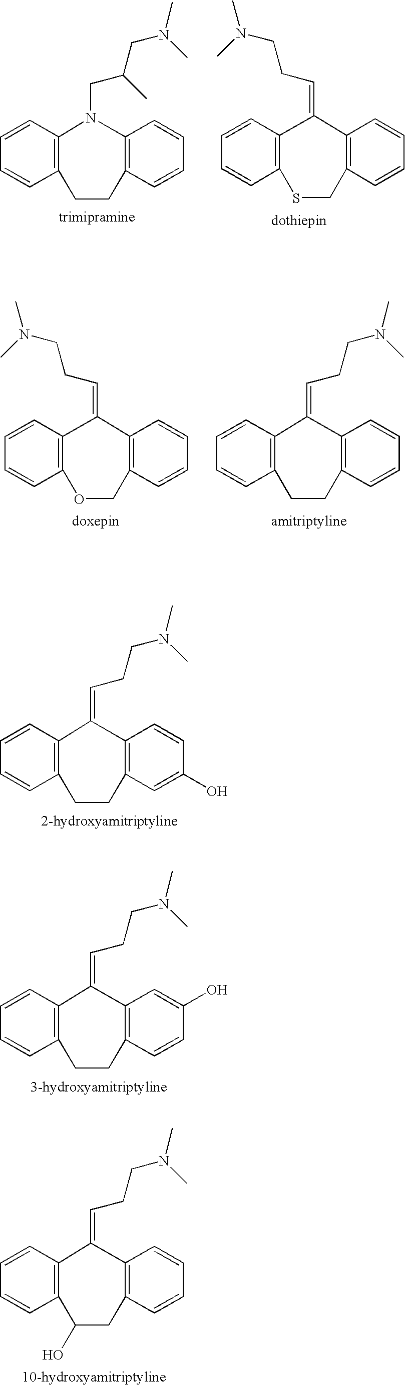

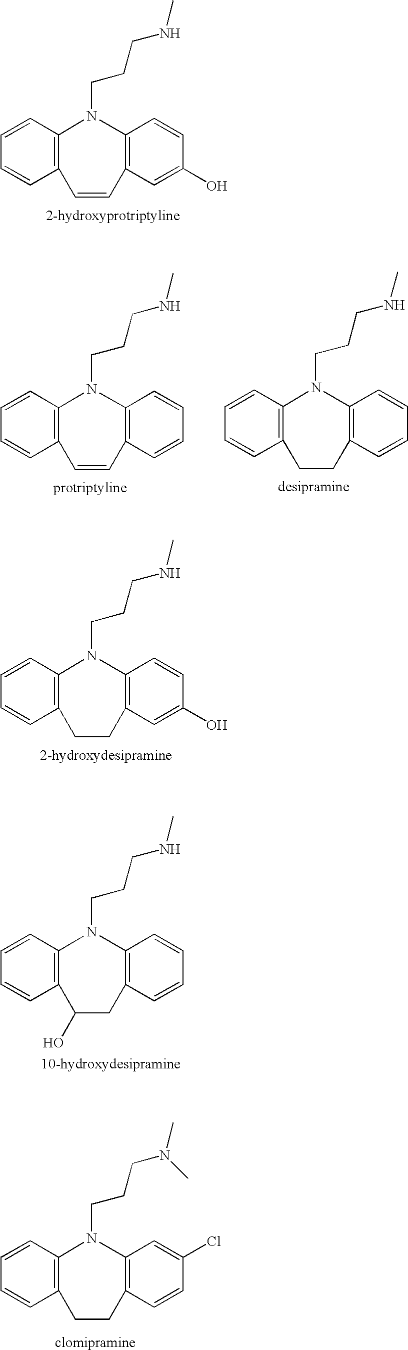

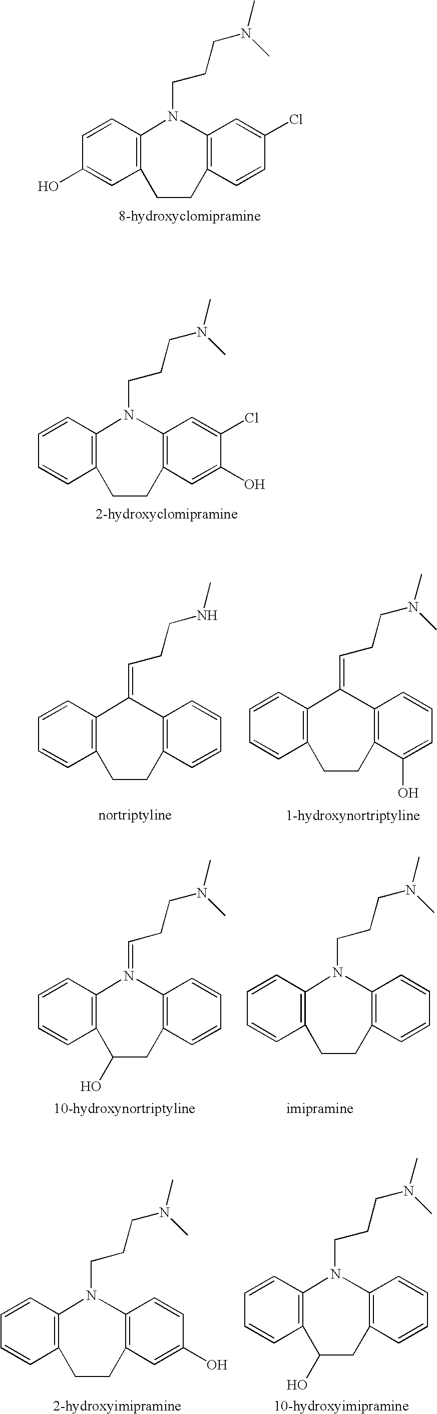

Selective Serotonin Reuptake Inhibitors (SSRIs) SSRIs include cericlamine, citalopram, clovoxamine, cyanodothiepin, dapoxetine, escitalopram, femoxetine, fluoxetine, fluvoxamine, ifoxetine, indalpine, indeloxazine, litoxetine, paroxetine, sertraline, tametraline, viqualine, and zimeldine.

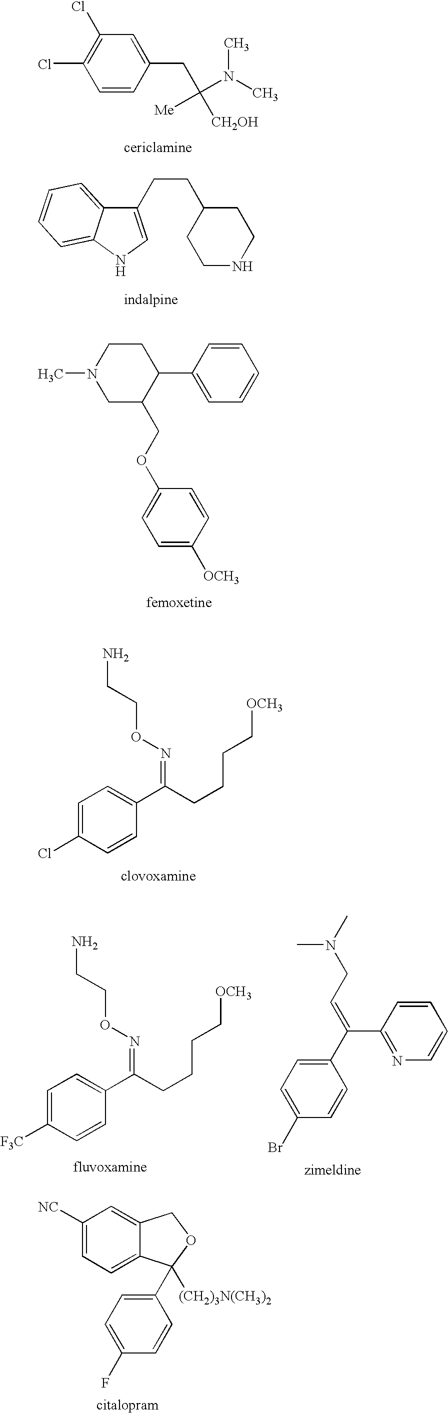

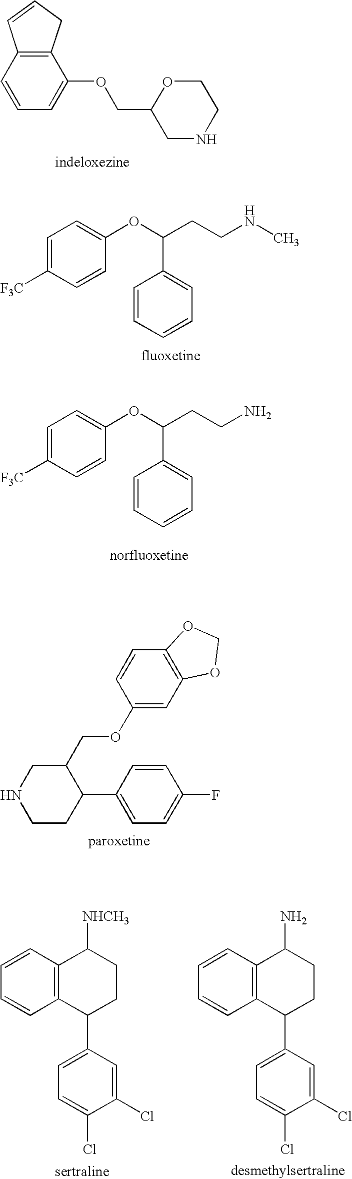

Serotonin Norepinephrine Reuptake Inhibitors (SNRIs)

SNRIs include milnacipran, venlafaxine and duloxetine. 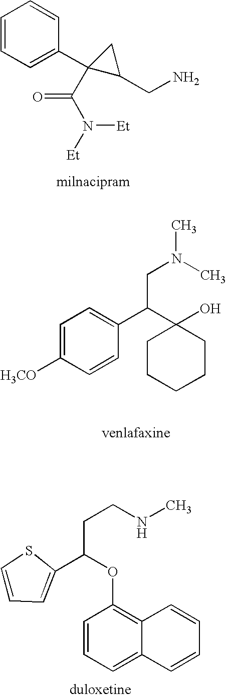
Linkers

The linker component of the invention is, at its simplest, a bond between a compound and a group that is bulky or charged. The linker provides a linear, cyclic, or branched molecular skeleton having pendant groups covalently linking a compound to a group that is bulky or charged.

Thus, the linking of a compound to a group that is bulky or charged is achieved by covalent means, involving bond formation with one or more functional groups located on the compound and the bulky or charged group. Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of a compound and a group that is bulky or charged may be effected using a linker which contains reactive moieties capable of reaction with such functional groups present in the compound and the bulky or charged group. For example, an amine group of the compound may react with a carboxyl group of the linker, or an activated derivative thereof, resulting in the formation of an amide linking the two.

Examples of moieties capable of reaction with sulfhydryl groups include α-haloacetyl compounds of the type XCH₂CO— (where X=Br, Cl or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulphide bridges.

Examples of reactive moieties capable of reaction with amino groups include, for example, alkylating and acylating agents. Representative alkylating agents include:
(i) α-haloacetyl compounds, which show specificity towards amino groups in the absence of reactive thiol groups and are of the type XCH₂CO— (where X=Cl, Br or I), for example, as described by Wong Biochemistry 24:5337 (1979);
(ii) N-maleimide derivatives, which may react with amino groups either through a Michael type reaction or through acylation by addition to the ring carbonyl group, for example, as described by Smyth et al., J. Am. Chem. Soc. 82:4600(1960) and Biochem. J 91:589 (1964);
(iii) aryl halides such as reactive nitrohaloaromatic compounds;
(iv) alkyl halides, as described, for example, by McKenzie et al., J. Protein Chem. 7:581 (1988);
(v) aldehydes and ketones capable of Schiff's base formation with amino groups, the adducts formed usually being stabilized through reduction to give a stable amine;
(vi) epoxide derivatives such as epichlorohydrin and bisoxiranes, which may react with amino, sulfhydryl, or phenolic hydroxyl groups;
(vii) chlorine-containing derivatives of s-triazines, which are very reactive towards nucleophiles such as amino, sufhydryl, and hydroxyl groups;
(viii) aziridines based on s-triazine compounds detailed above, e.g., as described by Ross, J. Adv. Cancer Res. 2:1 (1954), which react with nucleophiles such as amino groups by ring opening;
(ix) squaric acid diethyl esters as described by Tietze, Chem. Ber. 124:1215 (1991); and
(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:
(i) isocyanates and isothiocyanates, particularly aromatic derivatives, which form stable urea and thiourea derivatives respectively;
(ii) sulfonyl chlorides, which have been described by Herzig et al., Biopolymers 2:349 (1964);
(iii) acid halides;
(iv) active esters such as nitrophenylesters or N-hydroxysuccinimidyl esters;
(v) acid anhydrides such as mixed, symmetrical, or N-carboxyanhydrides;
(vi) other useful reagents for amide bond formation, for example, as described by M. Bodansky, Principles of Peptide Synthesis, Springer-Verlag, 1984;
(vii) acylazides, e.g. wherein the azide group is generated from a preformed hydrazide derivative using sodium nitrite, as described by Wetz et al., Anal. Biochem. 58:347 (1974); and
(viii) imidoesters, which form stable amidines on reaction with amino groups, for example, as described by Hunter and Ludwig, J. Am. Chem. Soc. 84:3491 (1962).

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in the compound and/or the bulky or charged group may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of the antidepressant with a reactive chemical group of the bulky or charged group without introducing additional linking material may, if desired, be used in accordance with the invention. For example, the amino group of an antidepressant can be converted to a sulfamic acid group (R—NH—S(O)₂(OH)). The sulfamic acid derivative is an anion at physiological pH.

Most commonly, however, the linker will include two or more reactive moieties, as described above, connected by a spacer element. The presence of such a spacer permits bifunctional linkers to react with specific functional groups within the antidepressant and the bulky or charged group, resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between the antidepressant and the bulky or charged group.

Spacer elements in the linker typically consist of linear or branched chains and may include a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl.

In some instances, the linker is described by formula (VII):
G¹-(Z¹)ₒ-(Y¹)ᵤ-(Z²)ₛ-(R₃₀)-(Z³)ₜ-(Y²)ᵥ-(Z⁴)ₚ-G² (VII)

In formula (VII), G¹ is a bond between the compound and the linker; G² is a bond between the linker and the bulky group or between the linker and the charged group; Z¹, Z², Z³, and Z⁴ each, independently, is selected from O, S, and NR₃₁; R₃₁ is hydrogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; Y¹ and Y² are each, independently, selected from carbonyl, thiocarbonyl, sulphonyl, or phosphoryl; o, p, s, t, u, and v are each, independently, 0 or 1; and R₃₀ is a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl, or a chemical bond linking G¹-(Z¹)ₒ-(Y¹)ᵤ-(Z²)ₛ- to -(Z³)ₜ(Y²)ᵥ-(Z⁴)ₚ-G².

Bulky Groups

The function of the bulky group is to increase the size of the compound sufficiently to inhibit passage across the blood-brain barrier. Bulky groups capable of inhibiting passage of the compound across the blood-brain barrier include those having a molecular weight greater than 300, 400, 500, 600, 700, 800, 900, or 1000 daltons. Desirably, these groups are attached through a nitrogen atom of the parent compound.

Corticosteroids

The bulky group may include a corticosteroid. Exemplary corticosteroids include, without limitation, hydrocortisone, budesonide, beclomethasone, methylprednisolone, prednisolone, prednisone, triamcinolone, dexamethasone, and betamethasone. The structures of several corticosteroids are provided below. Compounds conjugated to corticosteroid can be prepared by modification of an available functional group present in the parent corticosteroid. Typically, the corticosteroid is attached to a linker via an available hydroxyl group of the corticosteroid. Alternatively, an acyl or cyclic acetal group can be removed from the parent corticosteroid prior to conjugation to the compound. Accordingly, corticosteroids structurally related to those described herein can also be employed as a bulky group in the methods and compositions of the invention.

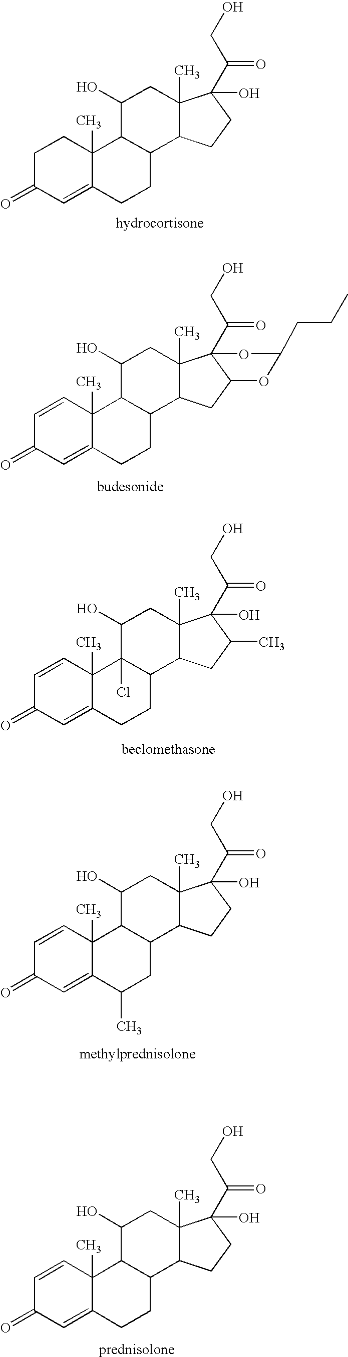

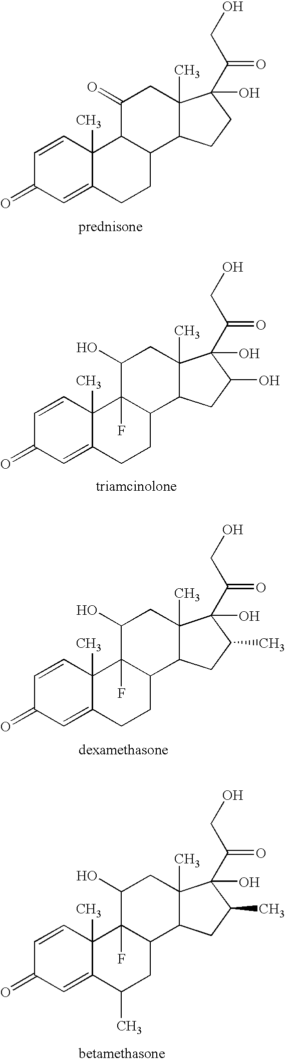

The bulky group may also be charged. For example, bulky groups include, without limitation, charged polypeptides, such as poly-arginine (guanidinium side chain), poly-lysine (ammonium side chain), poly-aspartic acid (carboxylate side chain), poly-glutamic acid (carboxlyate side chain), or poly-histidine (imidazolium side chain). An exemplary charged polysaccharide is hyaluronic acid (see below).

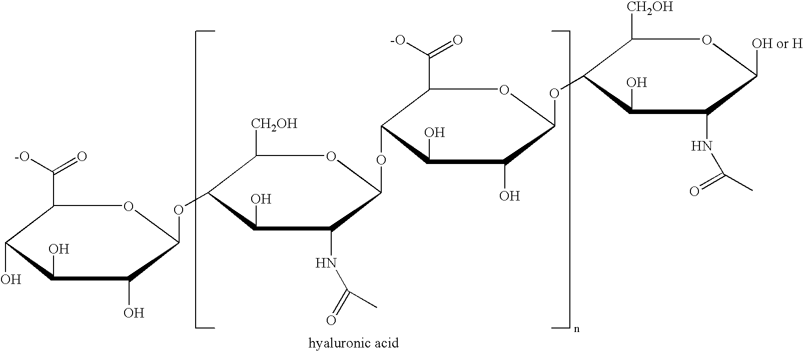

Desirably, a bulky group is selected which enhances the cellular uptake of the conjugate. For example, certain peptides enable active translocation across the plasma membrane into cells (e.g., RKKRRQRRR, the Tat (49-57) peptide). Exemplary peptides which promote cellular uptake are disclosed, for example, by Wender et al., Natl. Acad. Sci. USA 97:13003 (2000) and Laurent et al., FEBS Lett. 443:61 (1999), incorporated herein by reference. An example of a charged bulky group which facilitates cellular uptake is the polyguanidine peptoid (N-hxg)₉, shown below. Each of the nine guanidine side chains is a charged guanidinium cation at physiological pH. 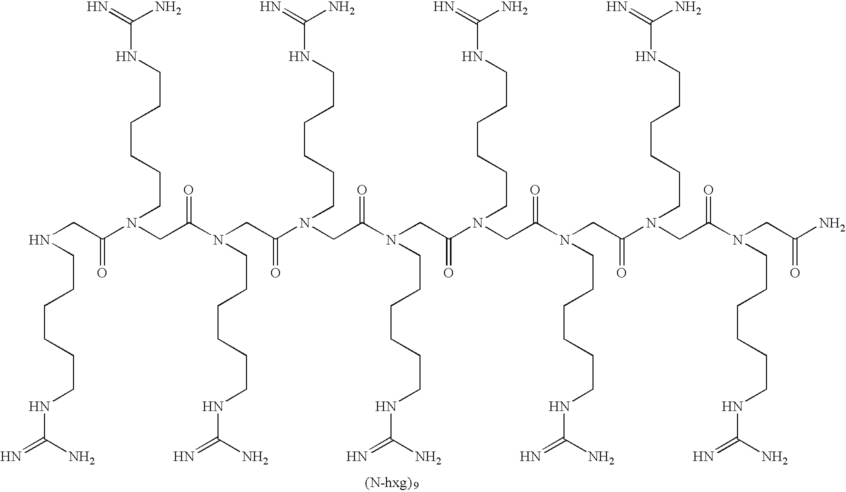
Charged Groups

The function of the charged group is to alter the charge of the compound sufficiently to inhibit passage across the blood-brain barrier. Desirably, charged groups are attached through a nitrogen atom of the parent compound.

A charged group may be cationic or an anionic. Charged groups include 1, 2, 3, 4, 5, 6, 7, 8, 9, 10, or more negatively charged moieties or 1, 2, 3, 4, 5, 6, 7, 8, 9, 10, or more positively charged moieties. Charged moieties include, without limitation, carboxylate, phosphodiester, phosphoramidate, borate, phosphate, phosphonate, phosphonate ester, sulfonate, sulfate, thiolate, phenolate, ammonium, amidinium, guanidinium, quaternary ammonium, and imidazolium moieties. For example, an ammonium group can be any amine that is protonated at physiological pH, such as in a morpholine ring.

Conjugates

The conjugates of the invention are designed to largely remain intact in vivo, resisting cleavage by intracellular and extracellular enzymes (e.g., amidases, esterases, and phosphatases). Any in vivo cleavage of the conjugate produces the parent compound, resulting in the unnecessary and potentially harmful exposure of the central nervous system to this compound. Thus, the conjugates of the invention are not prodrugs, but can be therapeutically active against immunoinflammatory disorders in their conjugated form, resulting in an improved therapeutic index relative to their parent, unconjugated, compound.

Conjugates may be further described by any one of formulas (XV)-(XXI):

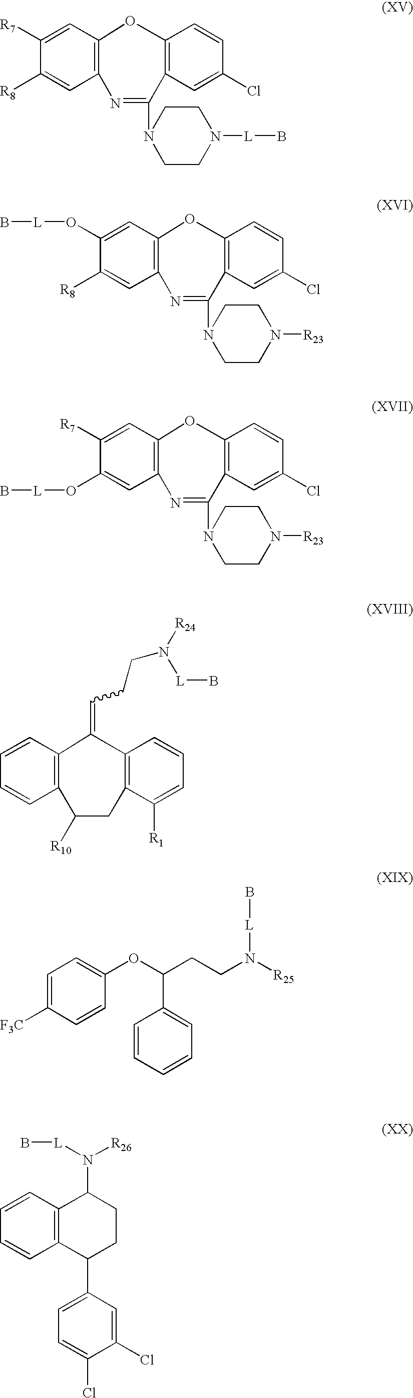

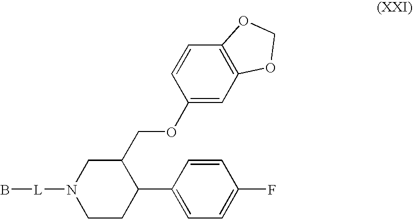

In formulas (XV)-(XXI), each of R₇ and R₈ is, independently, selected from H, and OH; each of R₂₃, R₂₄, R₂₅, and R₂₆ is, independently, selected from H and CH₃. L is a linker of formula (VII), described above. B is a bulky or charged group, as described above.

Conjugates can be prepared using techniques familiar to those skilled in the art. The conjugates can be prepared using the methods disclosed in, for example, G. Hermanson, Bioconjugate Techniques, Academic Press, Inc., 1996, as well as U.S. Pat. Nos. 2,779,775, 2,932,657, 4,472,392, 4,609,496, 4,820,700, 4,948,533, 4,950,659, 5,063,222, 5,215,979, 5,482,934, 5,939,409, and 6,140,308, each of which is incorporated herein by reference. Additional synthetic details are provided in the Examples.

Charge-Modified Antidepressants

The charge-modified antidepressants of the invention are not prodrugs, but can be therapeutically active against immunoinflammatory disorders in their charge-modified form, resulting in an improved therapeutic index relative to their parent, unmodified, antidepressant.

Charge-modified antidepressants include compounds of formulas (XXII)-(XXXI).

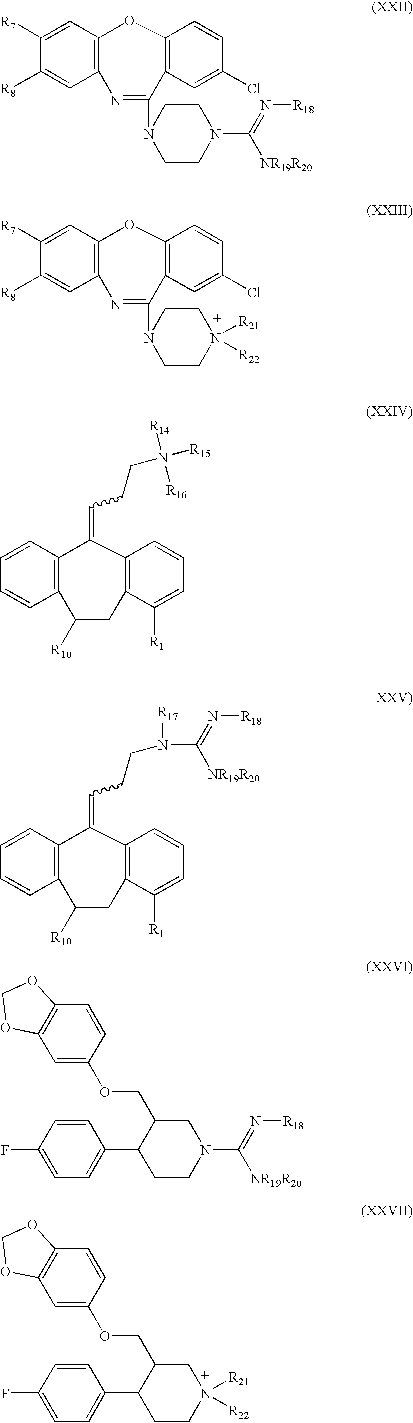

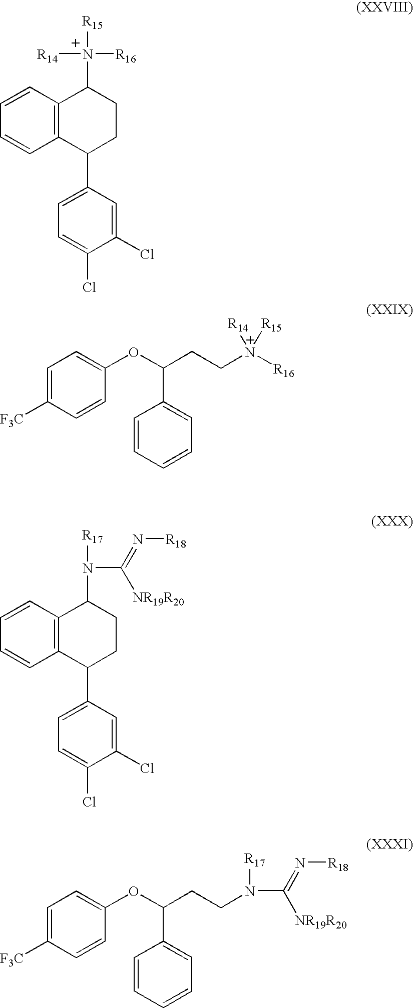

In formulas (XXII)-(XXIX), each of R₁, R₇, R₈, and R₁₀ is, independently, selected from H, and OH; each of R₁₄, R₁₅, R₁₆, R₂₁, and R₂₂ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C ₁₋₇ heteroalkyl; R₁₇ is H or CH₃; and each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms. Where R₁₈ and R₁₉ form a heterocyclic ring having two nitrogen atoms, the resulting guanidine group is, desirably, selected from 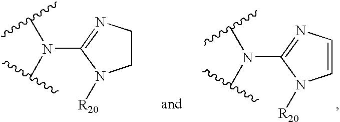
where R₂₀ is H or CH₃. Desirably, R₁₈ and R₁₉ combine to form an alkylene or alkenylene of from 2 to 4 carbon atoms, e.g., ring systems of 5, 6, and 7-membered rings. Such ring systems can be prepared, for example, using the methods disclosed by Schlama et al., J. Org. Chem., 62:4200 (1997).

Any of the antidepressants described herein can be modified as described above to form a charge-modified antidepressant having reduced CNS activity in comparison to the parent antidepressant.

Charge-modified antidepressants can be prepared using techniques familiar to those skilled in the art. The modifications can be made, for example, by alkylation of the parent antidepressants using the techniques described by J. March, Advanced Organic Chemistry: Reactions, Mechanisms and Structure, John Wiley & Sons, Inc., 1992, page 617. The conversion of amino groups to guanidine groups can be accomplished using standard synthetic protocols. For example, Mosher has described a general method for preparing mono-substituted guanidines by reaction of aminoiminomethanesulfonic acid with amines (Kim et al., Tetrahedron Lett. 29:3183 (1988)). A more convenient method for guanylation of primary and secondary amines was developed by Bernatowicz employing 1H-pyrazole-1-carboxamidine hydrochloride; 1-H-pyrazole-1-(N,N′-bis(tert-butoxycarbonyl)carboxamidine; or 1-H-pyrazole-1-(N,N′-bis(benzyloxycarbonyl)carboxamidine. These reagents react with amines to give mono-substituted guanidines (see Bernatowicz et al., J. Org. Chem. 57:2497 (1992); and Bernatowicz et al., Tetrahedron Lett. 34:3389 (1993)). In addition, Thioureas and S-alkyl-isothioureas have been shown to be useful intermediates in the syntheses of substituted guanidines (Poss et al., Tetrahedron Lett. 33:5933 (1992)). Additional synthetic details are provided in the Examples.

Assays

The compounds of the invention can be assayed by using standard in vitro models or animal models to evaluate their therapeutic activity. These assays are presently described in the literature and are familiar to those skilled in the art. Some of these are described below and in the Examples.

Compounds of the invention can be tested for the ability to suppress secretion of IFNγ, IL-1β, IL-2, and TNFα from stimulated white blood cells, and the percent inhibition of cytokine secretion, relative to untreated stimulated white blood cells, as described in U.S. Ser. No. 10/670,488, filed Sep. 24, 2003, and incorporated herein by reference.

The biodistribution of a conjugate can be measured by autoradiography. (see Example 9).

Therapy

Conjugates and charge-modified antidepressants can be administered locally or systemically to decrease inflammatory and immune responses. They can be used systemically in high doses in emergencies, for example, to treat anaphylactic reactions. They can be used in lower doses to treat inflammatory diseases including arthritis.

Therapeutic formulations may be in the form of liquid solutions or suspensions; for oral administration, formulations may be in the form of tablets or capsules; for ocular administration, formulations may be in the form of eye drops; for topical administration, formulations may be in the form of creams or lotions; and for intranasal formulations, in the form of powders, nasal drops, or aerosols.

Methods well known in the art for making formulations are found, for example, in “Remington: The Science and Practice of Pharmacy” (20th ed., ed. A. R. Gennaro, 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

Conjugates and charge-modified antidepressants may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, calcium, sodium, potassium and the like.

Administration of conjugates and charge-modified antidepressants in controlled release formulations is useful where the compound of formula I has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release of the conjugate or charge-modified antidepressant. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The invention features methods for modulating the immune response as a means for treating an immunoinflammatory disorder, proliferative skin disease, organ transplant rejection, or graft versus host disease.

Combination Therapy

The invention features methods for modulating the immune response as a means for treating an immunoinflammatory disorder, proliferative skin disease, organ transplant rejection, or graft versus host disease. The suppression of cytokine secretion can be achieved by administering a conjugate or charge-modified antidepresssant in combination with one or more steroids. Additional therapies are described below.

Chronic Obstructive Pulmonary Disease

In one embodiment, the methods, compositions, and kits of the invention are used for the treatment of chronic obstructive pulmonary disease (COPD). If desired, one or more agents typically used to treat COPD may be used as a substitute for or in addition to a corticosteroid in the methods, compositions, and kits of the invention. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline). Thus, in one embodiment, the invention features the combination of a conjugate or charge-modified antidepresssant and a bronchodilator, and methods of treating COPD therewith.

Psoriasis

The methods, compositions, and kits of the invention may be used for the treatment of psoriasis. If desired, one or more antipsoriatic agents typically used to treat psoriasis may be used as a substitute for or in addition to a corticosteroid in the methods, compositions, and kits of the invention. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, the invention features the combination of a conjugate or charge-modified antidepresssant and an antipsoriatic agent, and methods of treating psoriasis therewith.

Inflammatory Bowel Disease

The methods, compositions, and kits of the invention may be used for the treatment of inflammatory bowel disease. If desired, one or more agents typically used to treat inflammatory bowel disease may be used as a substitute for or in addition to a corticosteroid in the methods, compositions, and kits of the invention. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, the invention features the combination of a conjugate or charge-modified antidepresssant and any of the foregoing agents, and methods of treating inflammatory bowel disease therewith.

Rheumatoid Arthritis

The methods, compositions, and kits of the invention may be used for the treatment of rheumatoid arthritis. If desired, one or more agents typically used to treat rheumatoid arthritis may be used as a substitute for or in addition to a corticosteroid in the methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, the invention features the combination of a conjugate or charge-modified antidepresssant with any of the foregoing agents, and methods of treating rheumatoid arthritis therewith.

Asthma

The methods, compositions, and kits of the invention may be used for the treatment of asthma. If desired, one or more agents typically used to treat asthma may be used as a substitute for or in addition to a corticosteroid in the methods, compositions, and kits of the invention. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, the invention features the combination of a conjugate or charge-modified antidepresssant and any of the foregoing agents, and methods of treating asthma therewith.

Corticosteroids

If desired, one or more corticosteroids may be administered in a method of the invention or may be formulated with conjugates and charge-modified antidepressants in a composition of the invention. Suitable corticosteroids include 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone famesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

Standard recommended dosages for various steroid/disease combinations are provided in Table 1, below.

TABLE 1
Standard Recommended Corticosteroid Dosages

| Indication | Route | Drug | Dose | | Schedule |
|---|---|---|---|---|---|
| Psoriasis | oral | prednisolone | 7.5-60 | mg | per day or divided b.i.d. |
| | oral | prednisone | 7.5-60 | mg | per day or divided b.i.d. |
| Asthma | inhaled | beclomethasone dipropionate | 42 μg/puff) | | 4-8 puffs b.i.d. |
| | inhaled | budesonide | (200 μg/ | | 1-2 inhalations b.i.d. |
| | | | inhalation) | | |
| | inhaled | flunisolide | (250 μg/puff) | | 2-4 puffs b.i.d. |
| | inhaled | fluticasone propionate | (44, 110 or | | 2-4 puffs b.i.d. |
| | | | 220 μg/puff) | | |
| | inhaled | triamcinolone acetonide | (100 μg/puff) | | 2-4 puffs b.i.d. |
| COPD | oral | prednisone | 30-40 | mg | per day |
| Crohn's disease | oral | budesonide | 9 | mg | per day |
| Ulcerative colitis | oral | prednisone | 40-60 | mg | per day |
| | oral | hydrocortisone | 300 | mg (IV) | per day |
| | oral | methylprednisolone | 40-60 | mg | per day |
| Rheumatoid arthritis | oral | prednisone | 10 | mg | per day |

Other standard recommended dosages for corticosteroids are provided, e.g., in the Merck Manual of Diagnosis & Therapy (17th Ed. MH Beers et al., Merck & Co.) and Physicians' Desk Reference 2003 (57th Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone.

Steroid Receptor Modulators

Steroid receptor modulators (e.g., antagonists and agonists) may be used as a substitute for or in addition to a corticosteroid in the methods, compositions, and kits of the invention. Thus, in one embodiment, the invention features the combination of a conjugate or charge-modified antidepressant and a glucocorticoid receptor modulator or other steroid receptor modulator, and methods of treating immunoinflammatory disorders therewith.

Glucocorticoid receptor modulators that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO00/66522, each of which is hereby incorporated by reference. Other steroid receptor modulators may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used as a substitute for or in addition to a corticosteroid in the methods, compositions, and kits of the invention A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffinann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffinann-La Roche), ebiratide (Aventis), etiprednol dicloacetate (IVAX), fluazacort (Vicuron), flumoxonide (Hoffmnann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffinann-La Roche), tifluadom (Solvay), timobesone (Hoffmann-La Roche), TSC-5 (Takeda), and ZK-73634 (Schering AG).

Non-Steroidal Anti-Inflammatory Drugs (NSAIDs)

If desired, the conjugates and charge-modified antidepressants of the invention may be administered in conjunction with one or more of non-steroidal anti-inflammatory drugs (NSAIDs), such as naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin. The combination of a conjugate or charge-modified antidepressant with an NSAID can be more effective for the treatment of immunoinflammatory diseases, particulary those mediated by TNFα, IL-1, IL-2 or IFN-γ, than either agent alone.

Acetylsalicylic acid, also known by trade name aspirin, is useful in the relief of headache and muscle and joint aches. Aspirin is also effective in reducing fever, inflammation, and swelling and thus has been used for treatment of rheumatoid arthritis, rheumatic fever, and mild infection. Thus in one aspect, combination of a conjugate or charge-modified antidepressant and acetylsalicylic acid (aspirin) or an analog thereof can also be administered to enhance the treatment or prevention of any of the diseases mentioned above.

An NSAID may be administered in conjunction with any one of the combinations described in this application. For example, a patient suffering from immunoinflammatory disorder may be initially treated with a combination of a conjugate or charge-modified antidepressant and a corticosteroid and then treated with an NSAID, such as acetylsalicylic acid, in conjunction with the combination described above.

Dosage amounts of acetylsalicylic acid are known to those skilled in medical arts, and generally range from about 70 mg to about 350 mg per day. When a lower or a higher dose of aspirin is needed, a formulation containing dipyridamole and aspirin may contain 0-25 mg, 25-50 mg, 50-70 mg, 70-75 mg, 75-80 mg, 80-85 mg, 85-90 mg, 90-95 mg, 95-100 mg, 100-150 mg, 150-160 mg, 160-250 mg, 250-300 mg, 300-350 mg, or 350-1000 mg of aspirin.

When the combinations of the invention are used for treatment in conjunction with an NSAIDs it may be possible to reduce the dosage of the individual components substantially to a point below the dosages that would be required to achieve the same effects by administering NSAIDs (e.g., acetylsalicylic acid) or conjugate or charge-modified antidepressant alone or by administering a combination of an NSAID (e.g., acetylsalicylic acid) and a conjugate or charge-modified antidepressant.

In one aspect, the composition that includes a conjugate or charge-modified antidepressant and an NSAID has increased effectiveness, safety, tolerability, or satisfaction of treatment of a patient suffering from or at risk of suffering from immunoinflammatory disorder as compared to a composition having a conjugate or charge-modified antidepressant or an NSAID alone.

Nonsteroidal Immunophilin-Dependent Immunosuppressants

In one embodiment, the invention features methods, compositions, and kits employing a conjugate or charge-modified antidepressant and a non-steroidal immunophilin-dependent immunosuppressant (NsEDI), optionally with a corticosteroid or other agent described herein.

In healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus), and rapamycin target many types of immunoregulatory cells, including T-cells, and suppress the immune response in organ transplantation and autoimmune disorders.

In one embodiment, the NsIDI is cyclosporine, and is administered in an amount between 0.05 and 50 milligrams per kilogram per day (e.g., orally in an amount between 0.1 and 12 milligrams per kilogram per day). In another embodiment, the NsIDI is tacrolimus and is administered in an amount between 0.0001-20 milligrams per kilogram per day (e.g., orally in an amount between 0.01-0.2 milligrams per kilogram per day). In another embodiment, the NsIDI is rapamycin and is administered in an amount between 0.1-502 milligrams per day (e.g., at a single loading dose of 6 mg/day, followed by a 2 mg/day maintenance dose). In another embodiment, the NsIDI is everolimus, administered at a dosage of 0.75-8 mg/day. In still other embodiments, the NsIDI is pimecrolimus, administered in an amount between 0.1 and 200 milligrams per day (e.g., as a 1% cream/twice a day to treat atopic dermatitis or 60 mg a day for the treatment of psoriasis), or the NsIDI is a calcineurin-binding peptide administered in an amount and frequency sufficient to treat the patient. Two or more NsIDIs can be administered contemporaneously.

Cyclosporines

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar ((α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O-CH₂CH₂-OH)-8-Cs, and D-Ser-8-Cs, which are described in Cruz et al. (Antimicrob. Agents Chemother. 44:143-149, 2000).

Cyclosporines are highly hydrophobic and readily precipitate in the presence of water (e.g. on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

Cyclosporines can be administered either intravenously or orally, but oral administration is preferred. To overcome the hydrophobicity of cyclosporine A, an intravenous cyclosporine A may be provided in an ethanol-polyoxyethylated castor oil vehicle that must be diluted prior to administration. Cyclosporine A may be provided, e.g., as a microemulsion in a 25 mg or 100 mg tablets, or in a 100 mg/ml oral solution (NEORAL).

Typically, patient dosage of an oral cyclosporine varies according to the patient's condition, but some standard recommended dosages are provided herein. Patients undergoing organ transplant typically receive an initial dose of oral cyclosporine A in amounts between 12 and 15 mg/kg/day. Dosage is then gradually decreased by 5% per week until a 7-12 mg/kg/day maintenance dose is reached. For intravenous administration 2-6 mg/kg/day is preferred for most patients. For patients diagnosed as having Crohn's disease or ulcerative colitis, dosage amounts from 6-8 mg/kg/day are generally given. For patients diagnosed as having systemic lupus erythematosus, dosage amounts from 2.2-6.0 mg/kg/day are generally given. For psoriasis or rheumatoid arthritis, dosage amounts from 0.5-4 mg/kg/day are typical. A suggested dosing schedule is shown in Table 2. Other useful dosages include 0.5-5 mg/kg/day, 5-10 mg/kg/day, 10-15 mg/kg/day, 15-20 mg/kg/day, or 20-25 mg/kg/day. Often cyclosporines are administered in combination with other immunosuppressive agents, such as glucocorticoids.

TABLE 2

| | Atopic | | | | | | |
|---|---|---|---|---|---|---|---|
| Compound | Dermatitis | Psoriasis | RA | Crohn's | UC | Transplant | SLE |
| CsA | N/A | 0.5-4 mg/kg/ | 0.5-4 mg/kg/ | 6-8 mg/kg/ | 6-8 mg/kg/ | ˜7-12 mg/kg/ | 2.2-6.0 mg/kg/ |
| (NEORAL) | | day | day | day | day | day | day |
| | | | | (oral- | (oral) | | |
| | | | | fistulizing) | | | |
| Tacrolimus | 0.03-0.1% | 0.05-1.15 mg/kg/ | 1-3 mg/ | 0.1-0.2 mg/kg/ | 0.1-0.2 mg/kg/ | 0.1-0.2 mg/kg/ | N/A |
| | cream/twice | day | day | day | day | day | |
| | day (30 and | (oral) | (oral) | (oral) | (oral) | (oral) | |
| | 60 gram | | | | | | |
| | tubes) | | | | | | |
| Pimecrolimus | 1% | 40-60 mg/ | 40-60 mg/ | 80-160 mg/ | 160-240 mg/ | 40-120 mg/ | 40-120 mg/ |
| | cream/twice | day | day | day | day | day | day |
| | day (15, 30, | (oral) | (oral) | (oral) | (oral) | (oral) | (oral) |
| | 100 gram | | | | | | |
| | tubes) | | | | | | |

Table Legend
CsA = cyclosporine A
RA = rheumatoid arthritis
UC = ulcerative colitis
SLE = systemic lupus erythamatosus

Tacrolimus

Tacrolimus (FK506) is an immunosuppressive agent that targets T cell intracellular signal transduction pathways. Tacrolimus binds to an intracellular protein FK506 binding protein (FKBP-12) that is not structurally related to cyclophilin (Harding et al. Nature 341:758-7601, 1989; Siekienka et al. Nature 341:755-757, 1989; and Soltoff et al., J. Biol. Chem. 267:17472-17477, 1992). The FKBP/FK506 complex binds to calcineurin and inhibits calcineurin's phosphatase activity. This inhibition prevents the dephosphorylation and nuclear translocation of nuclear factor of activated T cells (NFAT), a nuclear component that initiates gene transcription required for proinflammatory cytokine (e.g., IL-2, gamma interferon) production and T cell activation. Thus, tacrolimus inhibits T cell activation.

Tacrolimus is a macrolide antibiotic that is produced by Streptomyces tsukubaensis. It suppresses the immune system and prolongs the survival of transplanted organs. It is currently available in oral and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 0.9% sodium chloride or 5% dextrose prior to injection. While oral administration is preferred, patients unable to take oral capsules may receive injectable tacrolimus. The initial dose should be administered no sooner than six hours after transplant by continuous intravenous infusion.

Tacrolimus and tacrolimus analogs are described by Tanaka et al., (J. Am. Chem. Soc., 109:5031, 1987) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

While suggested dosages will vary with a patient's condition, standard recommended dosages are provided below. Typically patients diagnosed as having Crohn's disease or ulcerative colitis are administered 0.1-0.2 mg/kg/day oral tacrolimus. Patients having a transplanted organ typically receive doses of 0.1-0.2 mg/kg/day of oral tacrolimus. Patients being treated for rheumatoid arthritis typically receive 1-3 mg/day oral tacrolimus. For the treatment of psoriasis, 0.01-0.15 mg/kg/day of oral tacrolimus is administered to a patient. Atopic dermatitis can be treated twice a day by applying a cream having 0.03-0.1% tacrolimus to the affected area. Patients receiving oral tacrolimus capsules typically receive the first dose no sooner than six hours after transplant, or eight to twelve hours after intravenous tacrolimus infusion was discontinued. Other suggested tacrolimus dosages include 0.005-0.01 mg/kg/day, 0.01-0.03 mg/kg/day, 0.03-0.05 mg/kg/day, 0.05-0.07 mg/kg/day, 0.07-0.10 mg/kg/day, 0.10-0.25 mg/kg/day, or 0.25-0.5 mg/kg/day.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus

Pimecrolimus is the 33-epi-chloro derivative of the macrolactam ascomyin. Pimecrolimus structural and functional analogs are described in U.S. Pat. No. 6,384,073. Pimecrolimus is particularly useful for the treatment of atopic dermatitis. Pimecrolimus is currently available as a 1% cream. Suggested dosing schedule for pimecrolimus is shown at Table 2. While individual dosing will vary with the patient's condition, some standard recommended dosages are provided below. Oral pimecrolimus can be given for the treatment of psoriasis or rheumatoid arthritis in amounts of 40-60 mg/day. For the treatment of Crohn's disease or ulcerative colitis amounts of 80-160 mg/day pimecrolimus can be given. Patients having an organ transplant can be administered 160-240 mg/day of pimecrolimus. Patients diagnosed as having systemic lupus erythamatosus can be administered 40-120 mg/day of pimecrolimus. Other useful dosages of pimecrolimus include 0.5-5 mg/day, 5-10 mg/day, 10-30 mg/day, 40-80 mg/day, 80-120 mg/day, or even 120-200 mg/day.

Rapamycin

Rapamycin is a cyclic lactone produced by Streptomyces hygroscopicus. Rapamycin is an immunosuppressive agent that inhibits T cell activation and proliferation. Like cyclosporines and tacrolimus, rapamycin forms a complex with the immunophilin FKBP-12, but the rapamycin-FKBP-12 complex does not inhibit calcineurin phosphatase activity. The rapamycin immunophilin complex binds to and inhibits the mammalian kinase target of rapamycin (mTOR). mTOR is a kinase that is required for cell-cycle progression. Inhibition of mTOR kinase activity blocks T cell activation and proinflammatory cytokine secretion.

Rapamycin structural and functional analogs include mono-and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Rapamycin is currently available for oral administration in liquid and tablet formulations. RAPAMUNE liquid contains 1 mg/mL rapamycin that is diluted in water or orange juice prior to administration. Tablets containing 1 or 2 mg of rapamycin are also available. Rapamycin is preferably given once daily as soon as possible after transplantation. It is absorbed rapidly and completely after oral administration. Typically, patient dosage of rapamycin varies according to the patient's condition, but some standard recommended dosages are provided below. The initial loading dose for rapamycin is 6 mg. Subsequent maintenance doses of 0.5-2 mg/day are typical. Alternatively, a loading dose of 3 mg, 5 mg, 10 mg, 15 mg, 20 mg, or 25 mg can be used with a 1 mg, 3 mg, 5 mg, 7 mg, or 10 mg per day maintenance dose. In patients weighing less than 40 kg, rapamycin dosages are typically adjusted based on body surface area; generally a 3 mg/m²/day loading dose and a 1 mg/m²/day maintenance dose is used.

Additional Applications

The compounds of the invention can be employed in immunomodulatory or mechanistic assays to determine whether other combinations, or single agents, are as effective as the combination in inhibiting secretion or production of proinflammatory cytokines or modulating immune response using assays generally known in the art, examples of which are described herein. For example, candidate compounds may be combined with a conjugate or charge-modified antidepressant or a corticosteroid and applied to stimulated PBMCs. After a suitable time, the cells are examined for cytokine secretion or production or other suitable immune response. The relative effects of the combinations versus each other, and versus the single agents are compared, and effective compounds and combinations are identified.

The combinations of the invention are also useful tools in elucidating mechanistic information about the biological pathways involved in inflammation. Such information can lead to the development of new combinations or single agents for inhibiting inflammation caused by proinflammatory cytokines. Methods known in the art to determine biological pathways can be used to determine the pathway, or network of pathways affected by contacting cells stimulated to produce proinflammatory cytokines with the compounds of the invention. Such methods can include, analyzing cellular constituents that are expressed or repressed after contact with the compounds of the invention as compared to untreated, positive or negative control compounds, and/or new single agents and combinations, or analyzing some other metabolic activity of the cell such as enzyme activity, nutrient uptake, and proliferation. Cellular components analyzed can include gene transcripts, and protein expression. Suitable methods can include standard biochemistry techniques, radiolabeling the compounds of the invention (e.g., ¹⁴C or ³H labeling), and observing the compounds binding to proteins, e.g. using 2d gels, gene expression profiling. Once identified, such compounds can be used in in vivo models to further validate the tool or develop new anti-inflammatory agents.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLE 1

### Protection and Deprotection of Reactive Groups

The synthesis of conjugates and charge-modified antidepressants may involve the selective protection and deprotection of alcohols, amines, ketones, sulfhydryls or carboxyl functional groups of the parent antidepressant, the linker, the bulky group, and/or the charged group. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994.

### EXAMPLE 2

### Preparation of Hydroxylated Tricyclic Antidepressants

The selective hydroxylation of tricyclic antidepressants can be achieved enzymatically using available methods. For example, in vitro methods for the hydroxylation of clomipramine, see Nielsen et al., J. Pharmacol. Exp. Ther. 277:1659 (1996); amitriptyline, see Zhang et al., Drug Metab. Dispos. 23:1417 (1995); doxepine, see Moody et al., Drug Metab. Dispos. 27:1157 (1999); and amoxapine, see Moody et al., Appl. Environ. Microbiol. 66:3646 (2000); have been described. The tricyclic antidepressant can be incubated in the presence of, for example, Cunninghamella elegans, liver microsomes, or P450 enzyme, e.g., CYP3A4 (Research Diagnostics, Inc., product number RDI-CYP3A4). The resulting mixture of hydroxylation products can be separated using HPLC. Alternatively, the hydroxylated tricyclic antidepressant can be prepared synthetically, for example, as described in Example 3.

### EXAMPLE 3

### Preparation 8-Hydroxyamoxapine

8-hydroxyamoxapine can be synthesized as shown in Scheme 1.

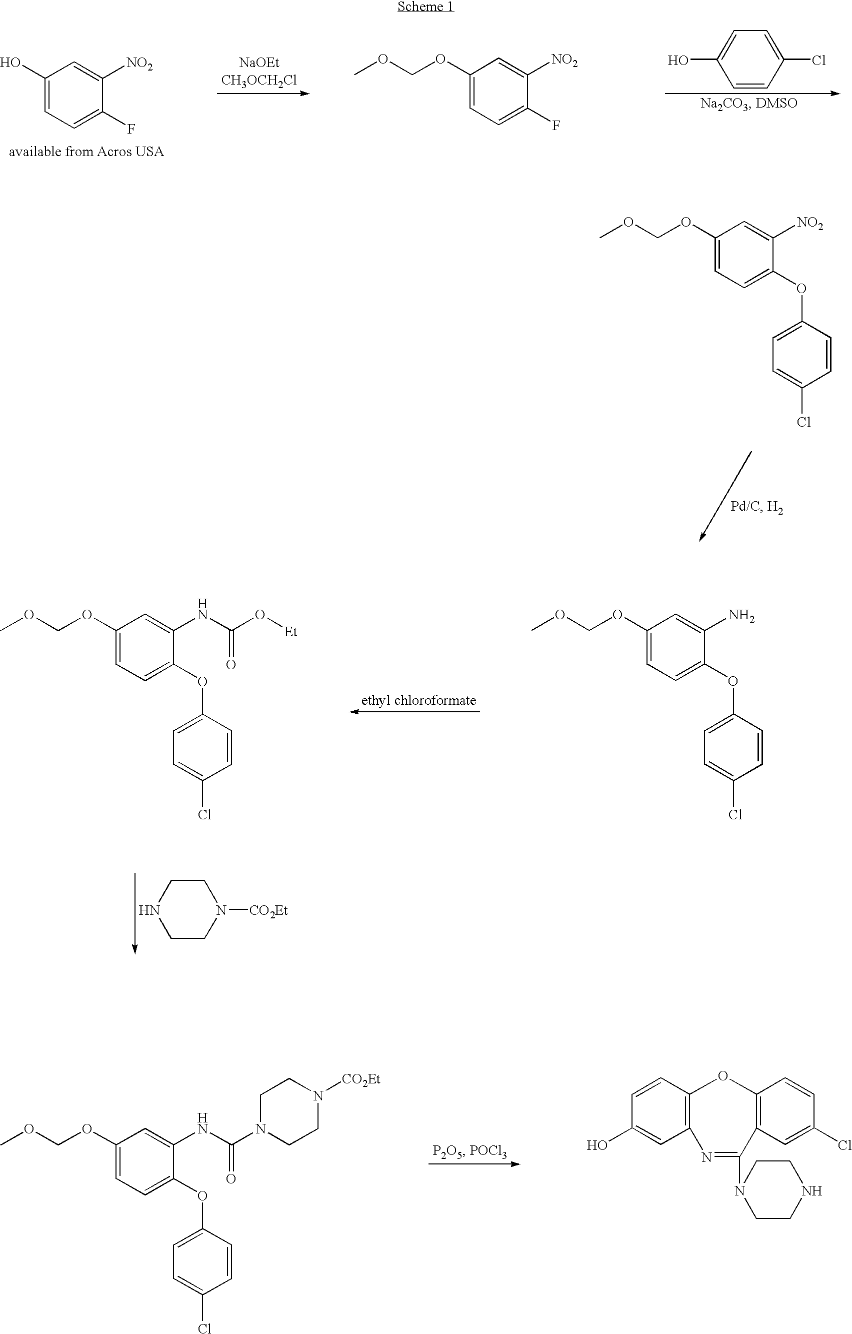

### EXAMPLE 4

### Preparation of Charge-Modified Antidepressants Including a Quatemized Amine

Charge-modified antidepressants can be prepared by alkylation of an amine nitrogen in the parent antidepressant as shown in Scheme 2. 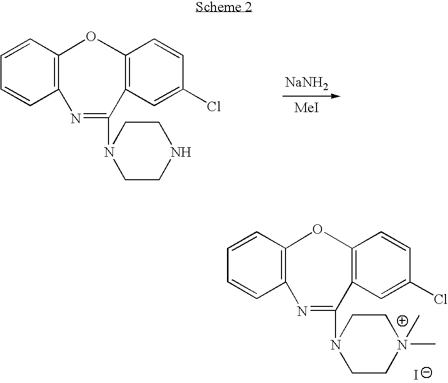
Any of the antidepressants described herein can be modified as shown in Scheme 2.

### EXAMPLE 5

### Preparation of Charge-Modified Antidepressants Including a Guanidine Group

The parent antidepressant can be reacted with a cynamide, e.g., methylcyanamide, as shown in Scheme 3. Alternatively, the parent antidepressant can be reacted with cyanogens bromide followed by reaction with methylchloroaluminum amide as shown in Scheme 4.

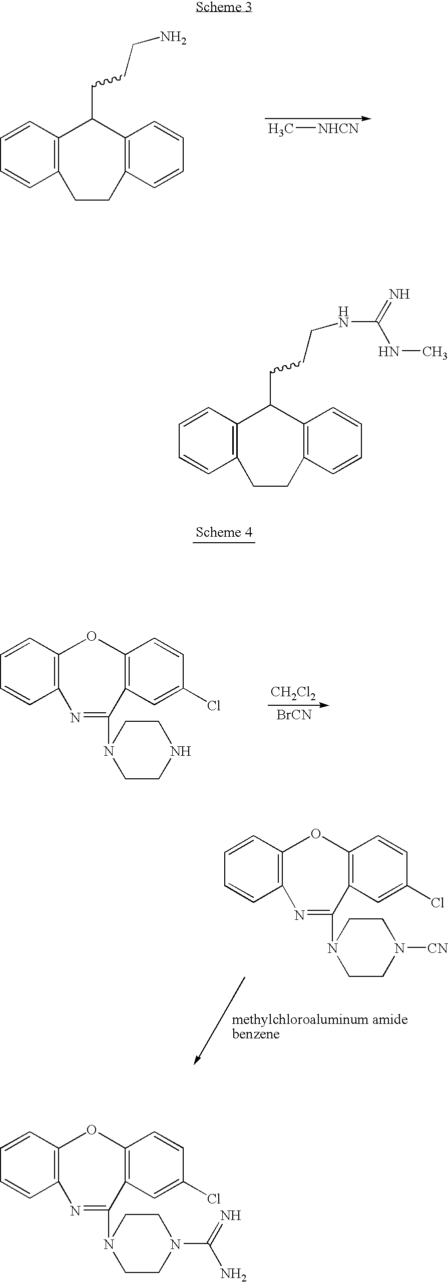

Any of the antidepressants described herein can be modified as shown in Schemes 3 and 4.

### EXAMPLE 5

### Preparation of a Compound Conjugated to an Anionic Group

A compound can be conjugated to an anionic group, e.g., carboxylate, as shown in Schemes 5 and 6, for amoxapine and 8-hydroxyamoxapine, respectively.

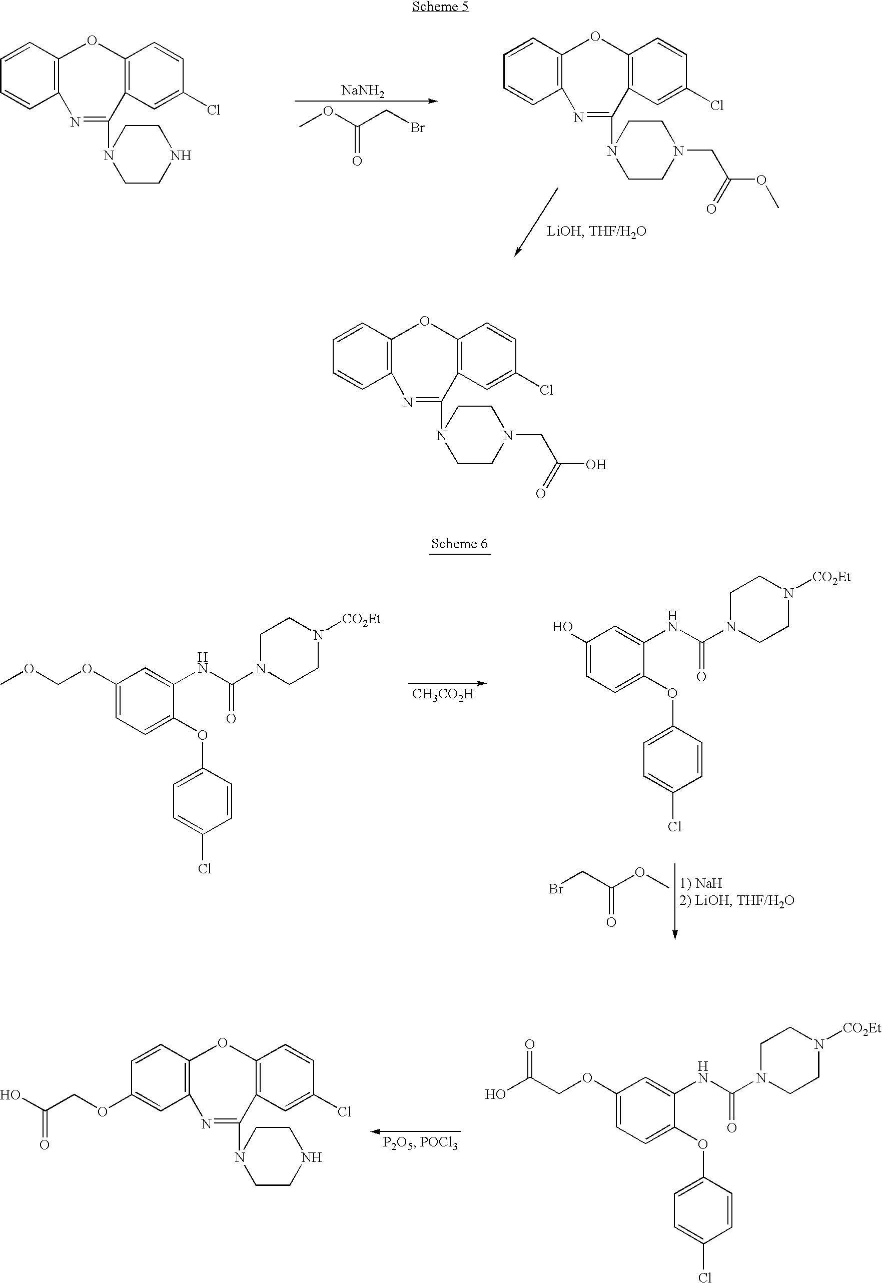

Any of the antidepressants described herein can be modified as shown in Schemes 5 and 6.

### EXAMPLE 6

### Preparation of a Compound Conjugated to a Cationic Group

A compound can be conjugated to a cationic group, e.g., morpholine, as shown in Scheme 7. 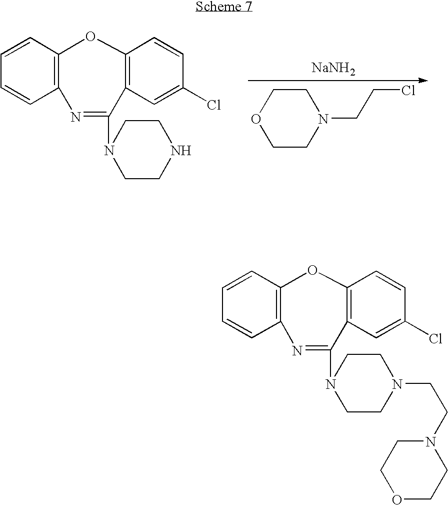
Any of the antidepressants described herein can be modified as shown in Scheme 7.

### EXAMPLE 7

### Preparation of a Compound Conjugated to a Bulky Group

A compound can be conjugated to a bulky group, e.g., PEG, as shown in Schemes 8 and 9.

The paroxetine can be conjugated to mono-methyl polyethylene glycol 5,000 propionic acid N-succinimidyl ester (Fluka, product number 85969) as shown in Scheme 4. The resulting mPEG conjugate, shown below, is an example of a compound conjugated to a bulky uncharged group.

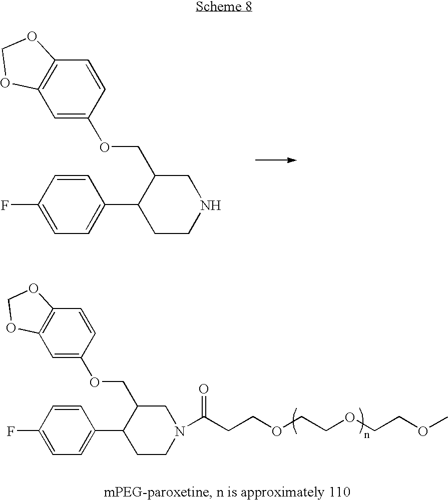

Conjugates of lower and higher molecular weight mPEG compounds can be prepared in a similar fashion.

The chemistry of Scheme 9 allows the PEG group, abbreviated RO—CH₂—Cl, to be attached via alkylation of an available hydroxy group. The resulting ether linkage is resistant to in vivo degradation.

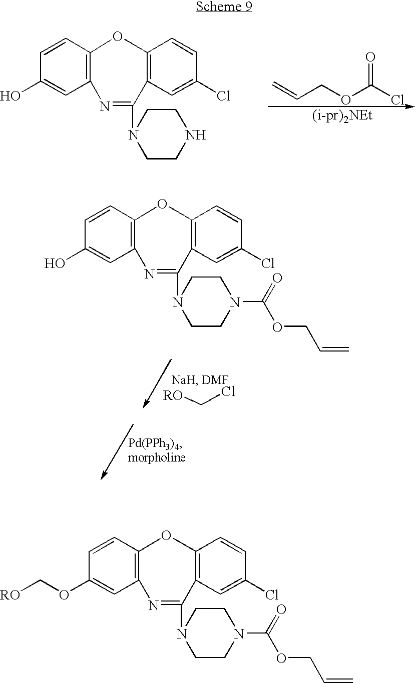

Any of the antidepressants described herein can be modified as shown in Schemes 8 and 9.

### EXAMPLE 8

### Preparation of Corticosteroid Conjugates

A compound can be conjugated to a bulky group, e.g., a corticosteroid, as shown in Scheme 10.

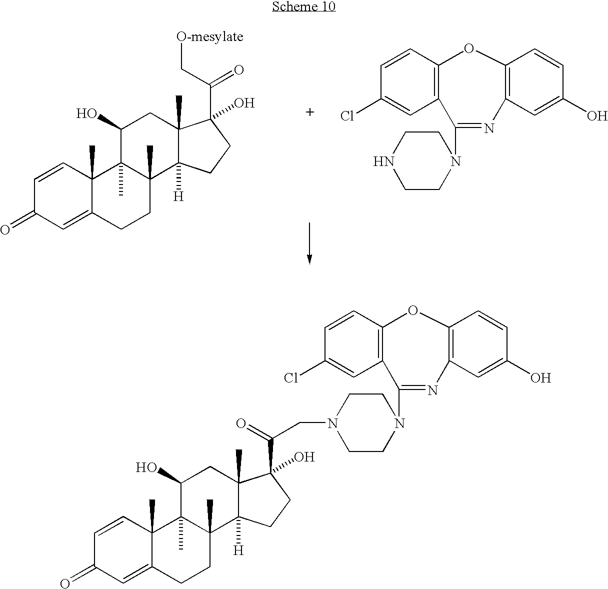

Any of the antidepressants described herein can be modified as shown in Scheme 10. 21-methanesulfonate prednisolone can be prepared according to the methods described in U.S. Pat. No. 2,932,657.

### EXAMPLE 9

### Autoradiography

The biodistribution of compounds of the invention can be assessed by in vivo autoradiography. In vivo autoradiography can be performed using ³H-labeled conjugates or ³H-labeled charge-modified antidepressants in adrenalectomized male Sprague-Dawley rats. First, the conjugate or charge-modified antidepressant is radioactively tagged, administered systemically to an adrenalectomized male Sprague-Dawley rat, and the animal is sacrificed. The brain is then rapidly removed and sliced into 10˜μm thick sections and mounted on slides. The slides are apposed to tritium-sensitive film, which is developed.

### EXAMPLE 9

### Lipopolysaccharide (LPS)-Induced Tumor Necrosis Factor Alpha (TNFα)

The purpose of this study was to examine the ability of compound 1 to suppress lipopolysaccharide (LPS)-induced Tumor Necrosis Factor alpha (TNFα) levels when administered to male Lewis rats.

Compound 1 was added directly to 100% ethanol. The suspension was vortexed vigorously until all of compound 1 was completely dissolved or fully suspended. The solution was made up to volume in 0.5% methylcellulose. The final concentration of ethanol was 10%.

At time minus 2 hours, rats were administered the appropriate amount of prednisolone and/or compound 1 via oral gavage in a dose volume of 0.25 mL. Untreated control and LPS Control animals received 0.25 mL of the vehicle only.

LPS was prepared at a 100X concentration by resuspension in Phosphate Buffered Saline (PBS). The LPS solution was vortexed vigorously to ensure complete suspension of the LPS. Immediately prior to injection, the LPS was serial diluted in PBS to a 1× working solution.

At time 0, animals were injected via the intraperitoneal route with 1.0 mL of the 1×LPS working solution (final LPS dose is 0.01 mg/Kg body weight) using a 25 gauge needle. Vehicle control animals received 1 mL of PBS.

Animals were euthanized via carbon dioxide asphyxiation and blood withdrawn from the inferior vena cava using a 27 gauge needle attached to a 3 mL syringe. Blood was expelled into a serum separator tube containing Clot Activator (Becton Dickinson, Franklin Lakes, N.J.). Samples were allowed to sit at room temperature for 30 min prior to spinning at 2000 rpm for 10 min in a bench top centrifuge at room temperature. Serum was transferred to an Eppendorf tube and immediately assayed for TNFα or stored at −80° C. until assayed.

Serum samples were assayed using the BioSource Rat TNFα ELISA kit according to the manufacturer's instructions.

Evaluation of the results included statistical analysis of differences in serum TNFα between treatment and control groups. Group means were compared using a one-way ANOVA. If the ANOVA was significant, p≦0.05, a multiple comparison test (Tukey-Kramer) was used to determine which groups were different. The results are summarized below in Table 3. The data show that compound 1 in combination with prednisolone suppresses LPS-induced TNFα.

TABLE 3

| Agent | Serum TNFα ± Standard Error |
|---|---|
| Vehicle | 867.7 ± 1042.1 |
| LPS | 23811.1 ± 7181.5 |
| 10 mg/kg prednisolone | 6239.3 ± 5020.4 |
| 0.3 mg/kg prednisolone | 11375.9 ± 5238.4 |
| 3 mg/kg compound1 | 12980.4 ± 6788.4 |
| 1 mg/kg compound1 | 12769.1 ± 5231.2 |
| 0.3 mg/kg compound1 | 13675.6 ± 7606.9 |
| 1 mg/kg compound1 and 0.3 mg/kg | 5394.0 ± 1786.7 |
| prednisolone | |

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A compound having the formula:
(A)-(L)-(B) ,
wherein
(B) is either a bulky group of greater than 300 daltons or a charged group of less than 300 daltons;
(L) is a linker which forms linkage groups with compound (A) and said group (B); and
(A) is a compound of formula I: 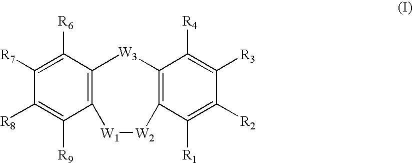
wherein
W₃ is O, CHCH₂R₅, or C═CHR₅;
W₁-W₂ is OCHR₁₁, SCHR₁₁, N═CR₁₁, CHR₁₀—CHR₁₁, or CR₁₀═CR₁₁;
each of R₁, R₂, R₃, R4, R₆, R₇, R₈, and R₉, is, independently, selected from H, OH, halide, and OG¹;
R₅ is CH₂CH₂X₁ or CH(CH₃)CH₂X₁;
R₁₀ is H, OH, or OG¹;
R₁₁ is H, OH, OG¹, or the group: 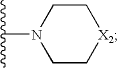
X₁ is NH₂, NHCH₃, N(CH₃)₂, NG¹(CH₃)₂, NG¹CH₃, or NHG¹;
X₂ is NH, NCH₃, NG ¹CH₃, or NG¹; and
G¹ is a bond in a linkage group between (A) and (L),
wherein said compound comprises one G¹, and
with the proviso that when (B) is a charged group of less than 300 daltons (B) does not comprise a carboxylic acid moiety.

2. The compound of claim 1 having formula II: 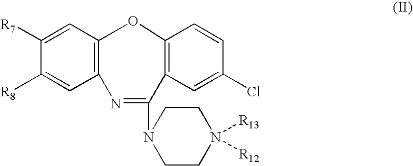
wherein
each of R₇ and R₈ is, independently, selected from H, OH, and OG¹;
R₁₂ is H, CH₃, or G¹; and
R₁₃ is CH₃ or absent.

3. The compound of claim 1 having formula III: 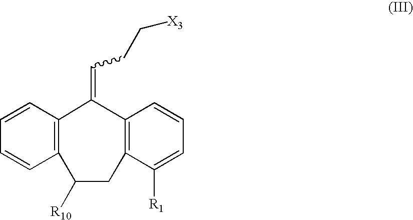
wherein
X₃ is NH₂, NHCH₃, N(CH₃)₂, NG¹(CH₃)₂, NG¹CH₃, or NHG¹; and
each of R₁ and R₁₀ is, independently, selected from H, OH, and OG¹.

4. The compound of claim 1, wherein said linker is described by formula VII:
G¹-(Z¹)ₒ-(Y¹)ᵤ-(Z²)ₛ-(R₃₀)-(Z³)ₜ-(Y²)ᵥ-(Z⁴)ₚ-G² (VII)
wherein
G¹ is the bond in a linkage group between said compound (A) and said linker;
G² is a bond in a linkage group between said linker and said bulky group or between said linker and said charged group;
Z¹, Z², Z³, and Z⁴ each, independently, is selected from O, S, and NR₃₁;
R₃₁ is hydrogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
Y¹ and Y² are each, independently, selected from carbonyl, thiocarbonyl, sulphonyl, or phosphoryl;
o, p, s, t, u, and v are each, independently, 0 or 1; and
R₃₀ is a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl, or a chemical bond linking G¹-(Z¹)ₒ-(Y¹)ᵤ-(Z²)ₛ- to -(Z³)ₜ-(Y²)ᵥ-(Z⁴)ₚ-G².

5. The compound of claim 1, wherein (B) is a bulky group of greater than 300 daltons and said bulky group comprises a naturally occurring polymer or a synthetic polymer.

6. The compound of claim 5, wherein said synthetic polymer is a polyethylene glycol.

7. The compound of claim 1, wherein (B) is a charged group of less than 300 daltons and said charged group is an anion.

8. The compound of claim 7, wherein said charged group comprises at least two negatively charged moieties.

9. The compound of claim 1, wherein said charged group is a cation.

10. The compound of claim 9, wherein said charged group comprises a morpholine ring.

11. The compound of claim 1, wherein (B) is a bulky group of greater than 300 daltons and said bulky group comprises a corticosteroid.

12. The compound of claim 11, wherein said corticosteroid is selected from hydrocortisone, methylprednisolone, prednisolone, prednisone, dexamethasone, budesonide, and triamcinolone.

13. A compound having the formula:
(A)-(L)-(B),
wherein
(B) is either a bulky group of greater than 300 daltons or a charged group of less than 300 daltons;
(L) is a linker which forms linkage groups with compound (A) and said group (B); and
(A) is a compound of formula IV: 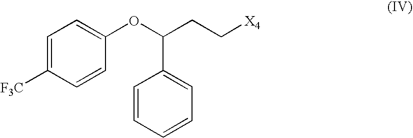
wherein
X₄ is NG¹(CH₃)₂, NG¹CH₃, or NHG¹; and
G¹ is a bond in a linkage group between (A) and (L).

14. A compound having the formula:
(A)-(L)-(B),
wherein
(B) is either a bulky group of greater than 300 daltons or a charged group of less than 300 daltons;
(L) is a linker which forms linkage groups with compound (A) and said group (B); and
(A) is a compound of formula V: 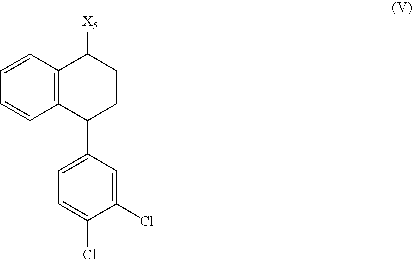
wherein
X₅ is NG¹(CH₃)₂, NG¹CH₃, or NHG¹; and
G¹ is a bond in a linkage group between (A) and (L).

15. A compound having the formula:
(A) -(L) -(B) ,
wherein
(B) is either a bulky group of greater than 300 daltons or a charged group of less than 300 daltons;
(L) is a linker which forms linkage groups with compound (A) and said group (B); and
(A) is a compound of formula VI: 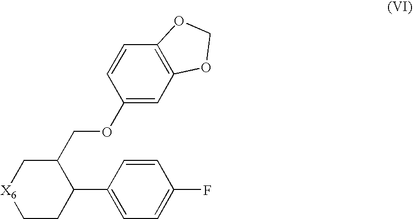
wherein
X₆ is NG¹CH₃, or NG¹; and
G¹ is a bond in a linkage group between (A) and (L).

16. A charge-modified antidepressant comprising a parent antidepressant having an amino nitrogen which been converted to a quaternary amino group or guanidinium group, and wherein said charge-modified antidepressant has anti-inflammatory activity in vivo and reduced activity in the central nervous system in comparison to said parent antidepressant.

17. The charge-modified antidepressant of claim 16, wherein said parent antidepressant is a tricyclic antidepressant.

18. The charge-modified antidepressant of claim 16, wherein said parent antidepressant is a selective serotonin reuptake inhibitor.

19. The charge-modified antidepressant of claim 16, wherein said parent antidepressant is a serotonin norepinephrine reuptake inhibitor.

20. The charge-modified antidepressant of claim 16 having formula VIII: 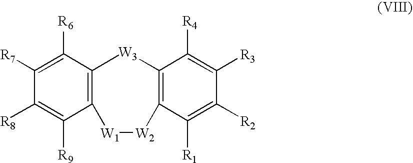
W₃ is O, CHCH₂R₅, or C═CHR₅;
W₁-W₂ is OCHR₁₁, SCHR, ₁₁, N═CR₁₁, CHR₁₀—CHR₁₁, or CR₁₀═CR₁₁;
each of R₁, R₂, R₃, R4, R₆, R₇, R₈, and R₉, is, independently, selected from H, OH, and halide;
R₅ is CH₂CH₂X₁, or CH(CH₃)CH₂X₁;
R₁₀ is H or OH;
R₁₁ is H, OH, or the group: 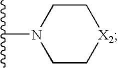
X₁ is NH₂, NHCH₃, N(CH₃)₂, NR₁₄R₁₅R₁₆, or NR₁₇X₇;
X₂ is NH, NCH₃, NR₂₁R₂₂, or NX₇;
each of R₁₄, R₁₅, R₁₆, R₂₁, and R₂₂ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl;
R₁₇ is H or CH₃;
X₇ is 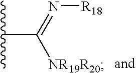
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

21. The charge-modified antidepressant of claim 20 having formula IX: 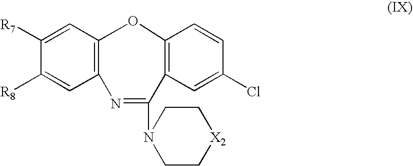
wherein
each of R₇ and R₈ is, independently, selected from H, and OH;
X₂ is NR₂₁R₂₂, or NX₇;
each of R₂₁, and R₂₂ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl;
X₇ is 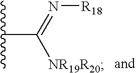
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

22. The charge-modified antidepressant of claim 20 having formula X: 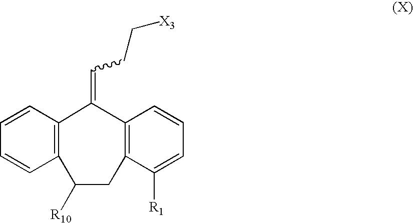
wherein
each of R₁ and R₁₀ is, independently, selected from H, and OH;
X₃ is NR₁₄R₁₅R₁₆, or NR₁₇X₇;
each of R₁₄, R₁₅, and R₁₆ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl;
R₁₇is H or CH₃;
X₇ is 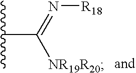
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

23. The charge-modified antidepressant of claim 16 having formula XI: 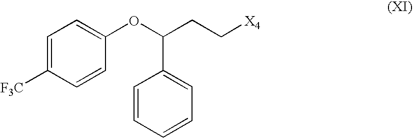
wherein
X₄ is NR₁₄R₁₅R₁₆, or NR₁₇X₇;
each of R₁₄, R₁₅, and R₁₆ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl;
R₁₇is H or CH₃;
X₇is 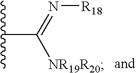
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

24. The charge-modified antidepressant of claim 16 having formula XII: 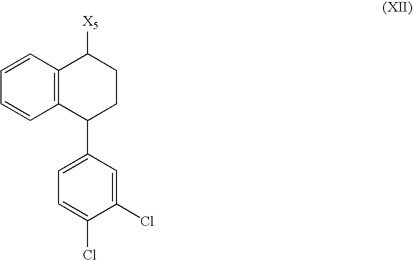
wherein
X₅ is NR₁₄R₁₅R₁₆, or NR₁₇X₇;
each of R₁₄, R₁₅, and R₁₆ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl;
R₁₇ is H or CH₃;
X₇ is 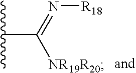
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

25. The charge-modified antidepressant of claim 16 having formula XIII: 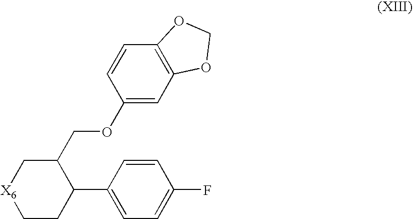
wherein
X₆ is NR₂₁R₂₂, or NX₇;
each of R₂₁, and R₂₂ is, independently, selected from C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₇ heteroalkyl;
X₇ is 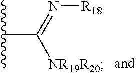
each of R₁₈, R₁₉, and R₂₀ is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, C₁₋₇ heteroalkyl, or R₁₈ and R₁₉ together complete a heterocyclic ring having two nitrogen atoms.

26. A method for suppressing secretion of one or more proinflammatory cytokines in a patient in need thereof, said method comprising administering to the patient a compound of any of claims 1-25 in an amount sufficient to suppress secretion of proinflammatory cytokines in said patient.

27. A method for treating a patient diagnosed with an immunoinflammatory disorder, said method comprising administering to the patient a compound of any of claims 1-25 in an amount sufficient to treat said patient.

28. A method of treating an inflammatory disorder in a patient, said method comprising administering to the patient a compound of any of claims 1-25 in an amount sufficient to treat said patient.

29. The method of claim 28, wherein said immunoinflammatory disorder is rheumatoid arthritis, osteoarthritis, Crohn's disease, ulcerative colitis, asthma, chronic obstructive pulmonary disease, polymyalgia rheumatica, giant cell arteritis, systemic lupus erythematosus, atopic dermatitis, multiple sclerosis, myasthenia gravis, psoriasis, ankylosing spondylitis, or psoriatic arthritis.

30. A method for treating a patient diagnosed with an immunoinflammatory disorder selected from rheumatoid arthritis, osteoarthritis, Crohn's disease, ulcerative colitis, chronic obstructive pulmonary disease, polymyalgia rheumatica, giant cell arteritis, systemic lupus erythematosus, atopic dermatitis, multiple sclerosis, myasthenia gravis, psoriasis, ankylosing spondylitis, and psoriatic arthritis, said method comprising administering to the patient a compound having the formula: 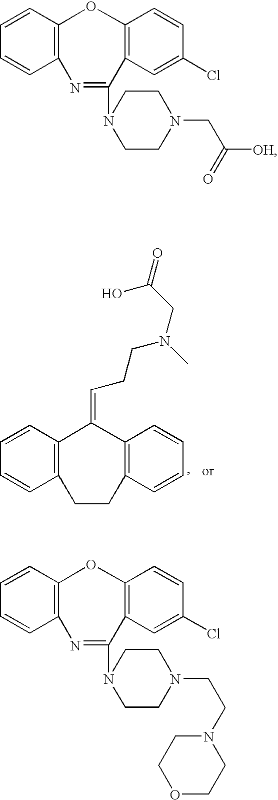
in an amount sufficient to treat said patient.

31. A method for inhibiting passage across the blood-brain barrier of a compound, said method comprising covalently attaching a group that is a bulky group of greater than 300 daltons or a charged group of less than 300 daltons, wherein said group increases the size, or alters the charge, of the compound sufficiently to inhibit passage across the blood-brain barrier without destroying the anti-inflammatory activity of said compound.

32. The method of claim 31, wherein said group is covalently linked via a nitrogen atom of said compound.

33. A method for inhibiting passage across the blood-brain barrier of a compound t having an amine nitrogen, said method comprising converting said amine nitrogen to a quaternary amino group or guanidinium group, wherein said group alters the charge of the compound sufficiently to inhibit passage across the blood-brain barrier without destroying the anti-inflammatory activity of said compound.

34. A pharmaceutical composition comprising an effective amount of a compound of any of claims 1-25, together with a pharmaceutically acceptable carrier or diluent.

35. A pharmaceutical composition comprising a compound of any of claims 1-25 and a corticosteroid in amounts that together are sufficient to treat an immunoinflammatory disorder when administered to a patient.

36. A pharmaceutical composition comprising:
(i) a compound having formula: 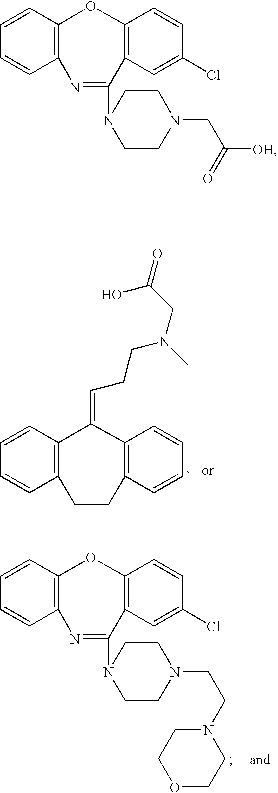
(ii) a corticosteroid,
wherein said compound and said corticosteroid are present in amounts that together are sufficient to treat an immunoinflammatory disorder when administered to a patient.

37. The pharmaceutical composition of claim 36, wherein said corticosteroid is prednisolone, cortisone, budesonide, dexamethasone, hydrocortisone, methylprednisolone, fluticasone, prednisone, triamcinolone, or diflorasone.

38. The pharmaceutical composition of claim 37, wherein said composition is formulated for topical administration.

39. The pharmaceutical composition of claim 37, wherein said composition is formulated for systemic administration.

40. A method of decreasing proinflammatory cytokine secretion or production in a patient, said method comprising administering to the patient a compound of any of claims 1-25 and a corticosteroid simultaneously or within 14 days of each other in an amount, that together, is sufficient to decrease proinflammatory cytokine secretion or production in said patient.

41. A method for treating a patient diagnosed with or at risk of developing an immunoinflammatory disorder, said method comprising administering to the patient a compound of any of claims 1-25 and a corticosteroid simultaneously or within 14 days of each other in amounts that together are sufficient to treat said patient.

42. The method of claim 41, wherein said immunoinflammatory disorder is rheumatoid arthritis, osteoarthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, or psoriatic arthritis.

43. A method for treating a patient diagnosed with or at risk of developing an immunoinflammatory disorder selected from rheumatoid arthritis, osteoarthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, and psoriatic arthritis, said method comprising administering to the patient:
(i) a compound having the formula: 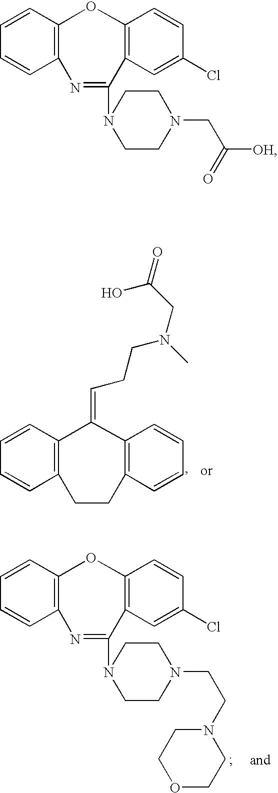
(ii) a corticosteroid,
wherein said compound and said corticosteroid are administered simultaneously or within 14 days of each other in amounts that together are sufficient to treat said patient.

44. The method of any of claim 43, wherein said corticosteroid is prednisolone, cortisone, budesonide, dexamethasone, hydrocortisone, methylprednisolone, fluticasone, prednisone, triamcinolone, or diflorasone.

45. A kit, comprising:
(i) a composition comprising a compound of any of claims 1-25 and a corticosteroid; and
(ii) instructions for administering said composition to a patient diagnosed with or at risk of developing an immunoinflammatory disorder.

46. A kit, comprising:
(i) a compound of any of claims 1-25;
(ii) a corticosteroid; and
(iii) instructions for systemically administering said compound and said corticosteroid to a patient diagnosed with or at risk of developing an immunoinflammatory disorder.

47. A kit comprising (i) a compound of any of claims 1-25 and (ii) instructions for administering said compound to a patient diagnosed with an immunoinflammatory disorder.

48. A kit comprising (i) a compound of any of claims 1-25 and (ii) instructions for administering said compound and a corticosteroid to a patient diagnosed with or at risk of developing an immunoinflammatory disorder.

49. A kit comprising (i) a corticosteroid and (ii) instructions for administering said corticosteroid and a compound of any of claims 1-25 to a patient diagnosed with or at risk of developing an immunoinflammatory disorder.

50. A kit, comprising:
(i) a compound having the formula: 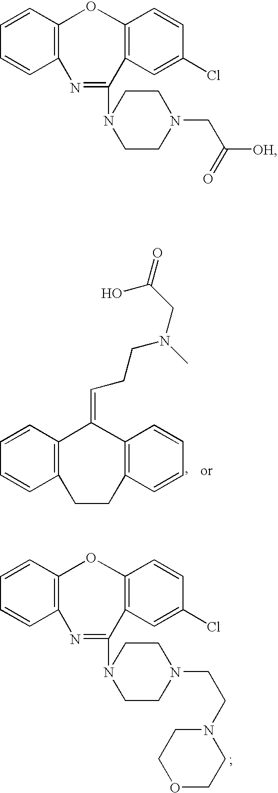
(ii) a corticosteroid; and
(iii) instructions for systemically administering said compound and said corticosteroid to a patient diagnosed with or at risk of developing an immunoinflammatory disorder.

51. A kit comprising:
(i) a compound having the formula: 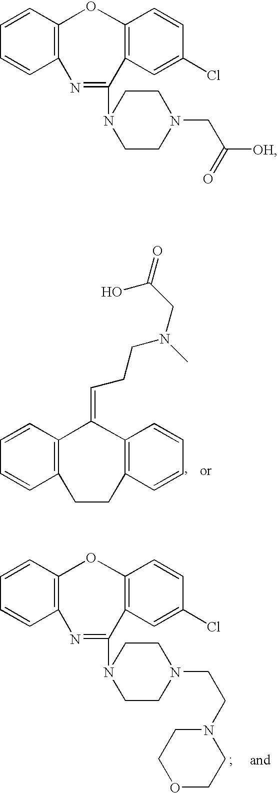
(ii) instructions for administering said compound to a patient diagnosed with an immunoinflammatory disorder selected from rheumatoid arthritis, osteoarthritis, Crohn's disease, ulcerative colitis, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, and psoriatic arthritis.
